(19) United States
(12) Patent Application Publication
(10) Pub. No.: US 2026/0240446 A1
(43) Pub. Date: Aug. 20, 2026

(54) UNIT, KIT AND SYSTEM FOR IDENTIFYING ABNORMALITIES IN A TISSUE OF A SUBJECT

(71) Applicant: Feminai LTD, Ramat Gan (IL)

(72) Inventors: Karny ILAN, Ramat Gan (IL); Gal YANUKA, Kibbutz Dalia (IL); Shani KLEIN ANTMAN, Tel Aviv-Yafo (IL)

(21) Appl. No.: 19/163,037

(22) PCT Filed: Mar. 7, 2024
(86) PCT No.: PCT/IL2024/050245

§ 371 Date: Sep. 8, 2025

(30) Foreign Application Priority Data
Mar. 8, 2023 (IL) 301229
Sep. 7, 2023 (IL) 305760

(51) Int. Cl. A61B 5/0531; A61B 5/00; A61B 5/01

(52) U.S. Cl. CPC A61B 5/0531; A61B 5/0091; A61B 5/015; A61B 5/4312; A61B 2562/046

(57) ABSTRACT

A system and method for identifying abnormalities in a tissue of a subject, particularly an abnormality related to cancer. A unit is configured for attachment to a portion of the breast and the armpit. The unit includes sensors configured to sense temperature and/or electrical profile responses from a skin portion following attachment of the unit. The unit further comprises at least one current electrode configured to apply electrical current to the skin such that the response to this electrical current profile is identified by the sensing spots. The electrical current can be through a first location and a sensing spot associated with a second location is configured to sense the response to the electrical current profile. Circuitry embedded in the unit is configured to (I) control the electrical current profile, (ii) operate the sensing spots, and (iii) transmit the sensed data that is measured by the sensing spots. A map of temperature profiles and electrical profile response of the breast can be generated and analyzed to identify signatures of tissue abnormality.

### TECHNOLOGICAL FIELD

The present disclosure is in the field of medical examination units, in particular a unit for examining a body part or a tissue in the body part of a subject for identifying abnormalities.

### BACKGROUND ART

References considered to be relevant as background to the presently disclosed subject matter are listed below:
CN108576952
US2019357774
KR200406517

Acknowledgement of the above references herein is not to be inferred as meaning that these are in any way relevant to the patentability of the presently disclosed subject matter.

### GENERAL DESCRIPTION

The present disclosure provides a unit, kit, system and method for identifying abnormalities in a tissue of a subject, particularly for identifying an abnormality related to cancer. The present disclosure makes use of a unit that is configured for attachment to a portion of the breast and optionally also to the armpit. Therefore, in one embodiment, the unit is configured to attach to the breast and the armpit as a single piece; and in a second embodiment, there are two sub-units, each is configured to independently attach to the breast and the armpit, respectively, and to communicate between them to transfer data. The unit includes sensing units or sensing spots that are configured to sense temperature or electrical profile response or both temperature and electrical profile response from a skin portion they are associated with following attachment of the unit, namely the skin portions that are in contact with or in proximity to the sensing units or sensing spots. The unit further comprises at least one current electrode that is configured to apply electrical current profile to the skin of the subject such that the response to this electrical current profile is identified by the sensing spots. Namely, the application of the electrical current can be through a first location of the skin of the subject and a sensing spot associated with a second location of the skin of the subject is configured to sense the response to the electrical current profile. A processing circuitry that is embedded in the unit is configured to (i) control the electrical current profile to execute a selected electrical current profile, (ii) operate the sensing spots to perform their measurements, and (iii) transmit the sensed data that is measured by the sensing spots. Based on the measurements of the sensing units, a map of temporal temperature profiles and electrical profile response of different portions of the breast can be generated. The temporal profiles of temperatures and electrical profile-related parameters are then analyzed to identify signatures of tissue abnormality.

Therefore, a first aspect of the present disclosure provides an examination unit for performing examination of a breast of a subject, and optionally an axilla of the subject, for identifying a tissue abnormality, e.g. a potential cancer tumor. The examination unit comprising: at least one attachable sheet configured for being attached to a first selected portion and, optionally a second selected portion, corresponding to at least one breast and one optionally axilla, respectively. Namely, the examination unit may be configured for measuring from the breast alone or from the breast and the axilla simultaneously. The attachable sheet comprises a plurality of measuring electrodes; a plurality of first sensing spots, each sensing spot configured for sensing from a tissue portion associated therewith either (i) electrical profile by a respective measuring electrode from said plurality of measuring electrodes; (ii) IR radiation profile or thermal profile; or (iii) both (i) electrical profile and (ii) IR radiation profile or thermal profile, and generate a respective first sensed data based thereon; at least one current electrode configured for applying electrical current to at least a tissue portion associated with a respective said first sensing spot, the electric current electrode and the measured electrode may have the same structure. The examination unit further comprises a processing circuitry; one or more memories coupled to the processing circuitry; and storing programming instructions for execution by the processing circuitry for: (1) operating said first and second sensing spots and receiving each of said respective first and second sensed data; (2) controlling said at least one current electrode to execute a selected electrical current profile; and (3) transmitting each of said respective first and second sensed data.

It is to be noted that any combination of the described embodiments with respect to any aspect of this present disclosure is applicable. In other words, any aspect of the present disclosure can be defined by any combination of the described embodiments.

In some embodiments of the examination unit, said at least one attachable sheet comprises a skin facing face, said skin facing face comprises an adhesive layer for allowing attachment to said first and second selected portions.

In some embodiments of the examination unit, said skin facing face comprises a removable cover covering said adhesive layer and configured for removal prior to said attachment.

In some embodiments of the examination unit, said at least one attachable sheet has a shape of a closed shape.

In some embodiments of the examination unit, said first selected portion comprises a majority of the breast area of said breast. The breast area is defined as the area spanning between the clavicle bone and mammary fold.

In some embodiments of the examination unit, said second selected portion comprises a majority of the axilla surface of said axilla. The axilla surface's anatomical borders in which the examination unit is to be placed are: (1) Anterior-Pectoralis major muscle (known as the axillary fold); (2) Posterior-latissimus dorsi muscle; (3) Superior-intertubercle sulcus of humerus; and (4) Inferior-lateral inframammary fold.

In some embodiments of the examination unit, said first selected portion and said second selected portion form a physical continuum being attachable to at least one said attachable sheet.

In some embodiments of the examination unit, said at least one attachable sheet comprises a first attachable sheet attachable to said first selected portion and a second attachable sheet attachable to said second selected portion, wherein said first and second attachable sheets are physically discrete. Namely, the two sheets are physically separated and can communicate between them for data exchange of sensing data, sensing profile execution data or both.

In some embodiments of the examination unit, the at least one attachable sheet is configured for being attached to the first selected portion and the second selected portion. Namely, the attachable sheet is attached to both the axilla portion of the subject and the breast of the subject to allow performing measurements from these portions. The at least one attachable sheet is a single unit that covers both portion.

In some embodiments, the examination unit further comprises a plurality of second sensing spots, each second sensing spot configured for sensing from a tissue portion associated therewith an IR radiation profile or thermal profile (both can be referred together as thermal profile) and generate a respective second sensed data based thereon.

In some embodiments of the examination unit, at least a portion of the measuring electrodes constitute said at least one current electrode. Namely, the measuring electrode is capable of executing the desired current profile to be applied to the skin portion it is associated with and also measuring the electrical profile in response to the execution of said current profile.

In some embodiments, the examination unit further comprises a proportion of (1:1) of measuring electrodes and thermal sensors in order to create overlapping maps for analysis.

In some embodiments of the examination unit, said electrical profile is determined by any of impedance, conductivity, electrical potential of the tissue, and permittivity, or any combination thereof, of the at least said tissue portion being associated with a respective first sensing spot thereto.

In some embodiments of the examination unit, said first sensing spots comprise a measuring electrode and a thermal sensor, whereby said electrode and said sensor are physically adjacent one to the other in each sensing spot. Namely, the thermal sensor and the measuring electrode are placed practically in the same place so that the data maps overlap.

In some embodiments of the examination unit, each of the plurality of first sensing spots comprises a measuring electrode and a thermal sensor, and wherein said first sensing spot is configured for sensing both of (i) electrical profile; and (ii) IR radiation profile or thermal profile of the respective tissue portion associated therewith. It is to be noted that each sensing spot is defined by a certain area that includes at least one thermal sensor and at least one measuring electrode. By arranging the sensors in this manner, the generated maps of the electrical profile and the thermal profile can be correlated easily.

In some embodiments of the examination unit, said at least one attachable sheet comprises a first sensing array having a plurality of said first sensing spots; each said sensing spot being positioned one with respect to the other along at least one first continuous line. In some embodiments, the distance between the sensors is equal or smaller than 5 mm in a constant-even distribution.

In some embodiments of the examination unit, said at least one first continuous line is configured to accommodate the shape and dimensions of said first selected portion.

In some embodiments of the examination unit, at least one of said continuous line defines a left-hand or right-hand spiral comprising said first sensing spots being arranged in a sequential and ordered manner and configured for permitting sensing of a majority of said selected first portion. The spirals are tailor made based on the universal breast cup sizes to ensure fit for all types of breasts. Also, the spiral shape is cut from a breast 3D scan or a breast shape database. The shape is cut to spirals to create a demo shaped in a spiral structure that fits the breast. A spiral shape can be geometrically defined as a curve that starts from a central point (nipple) and progressively moves outward or inward in a circular or helical pattern. The defining characteristic of a spiral is that it continuously expands or contracts while maintaining a constant distance between successive turns. The spiral arrangement of the sensor allows the sheet and the PCB that carries the sensors to be sufficiently flexible so as to conform with the breast shape.

In some embodiments of the examination unit, said continuous line forms a closed shape.

In some embodiments of the examination unit, said first sensing array comprises a plurality of said closed shapes arranged as concentric circles. For example, the center of these concentric circles may be about the position of a nipple placement position of said attachable sheet.

In some embodiments of the examination unit, the distance between each concentric circle, defined as R₁-R₂, wherein O₁>O₂, is equal or not equal but maintains a constant ratio throughout.

In some embodiments, the examination unit further comprises an optical sensor configured for identifying an anatomical mark of the breast and generate optical sensed data, either by its own processor or by the processing circuitry of the examination unit that receives the optical measurements of the optical sensor and analyze them to identify the anatomical mark, e.g. a nipple of the subject. The processing circuitry is configured for identifying a misplacement of the examination unit based on the optical sensed data and outputting an alert if a misplacement is identified.

In some embodiments, the examination unit further comprises one or more optical sensors configured for sensing optical parameters indicative of intensity profile of light of selected one or more wavelength ranges sensed in each optical sensor and to generate optical sensed data based thereon, either by its own processor or by the processing circuitry of the examination unit that receives the optical measurements of the optical sensor. The at least one processing circuitry is configured for analyzing said optical sensed data and identifying at least one of: (1) inadequate level of adhesiveness of the attachable sheet and/or stability thereof throughout the examination process (including the first step of attachment of the examination unit); and (2) misplacement of the examination unit. The processing circuitry is further configured and outputting an alert if (1) or (2) or both is identified.

In some embodiments of the examination unit, the one or more optical sensors are arranged along different portions of a periphery of the at least one attachable sheet.

In some embodiments of the examination unit, at least one of said continuous line defines an open-shape configuration configured for permitting sensing of a majority of said selected second portion.

In some embodiments of the examination unit, said at least one current electrode comprise one or more pairs of electrodes that are positioned in opposite sides of the attachable sheet, namely distanced one from the other, each member of said one or more pairs of electrodes is associated with a respective fat measuring electrode that is comprised in said plurality of measuring electrodes, namely the fat measuring electrode may also serve as a standard measuring electrode. The processing circuitry is configured to operate the one or more pairs of electrodes to apply a selected current profile to flow from a first member electrode of the pair to a second member of the pair. The electrical profile that is sensed by the respective fat measuring electrodes is indicative of the fat percentage in the tissue. Typically, the electrical current and the voltage between the two fat measuring electrodes is sensed and analyzed to extract the fat percentage of the breast tissue. This measurement may be used in order to analyze the generated maps and to contribute to the conclusion whether an abnormality is identified in the breast or axilla or not.

In some embodiments of the examination unit, said first sensing spots comprise said second sensing spots. Namely, the second sensing spots can also serve for performing the substantial measurements and not only for placement verification.

In some embodiments, the examination unit further comprises a second sensing array having a plurality of said second sensing spots, that can be part of the first array or different than the first array. Each second sensing spot being positioned one with respect to the other along at least one second continuous line, wherein the processing circuitry is further configured to analyze said second sensing data and to identify a heat signature of one or more anatomical landmarks for verifying a good placement of said attachable sheet, and if no heat signature is identified, the processing circuitry is configured to execute a misplacement alert. The landmarks are typically one or both of the internal mammary artery and the lateral thoracic artery of each breast. It is to be noted that when the at least one attachable sheet is a single sheet, a good placement verification by one of these arteries may also indicate a good placement on the axilla portion. In some embodiments, it may further require a simple verification that the axilla portion of the attachable sheet is indeed attached to the skin of the subject.

In some embodiments of the examination unit, said at least one second continuous line is generally vertical oriented if examined when the attachable sheet is correctly attached to the breast.

In some embodiments of the examination unit, said at least one second continuous line is disposed laterally to said first selected portion. For validation of correct placement of the breast unit: the lateral and medial borders of the device contain a vertical line of heat sensors and optionally optic sensors, aimed at detecting the lateral thoracic artery, nipple, and the internal mammary artery, thus assuring that the device is in place.

In some embodiments of the examination unit, said at least one second continuous line is medial to said first selected portion.

In some embodiments of the examination unit, said attachable sheet comprises a first layer and a second layer, and wherein the first layer comprises said first and second sensing arrays and at least one current electrode; and the second layer comprises said processing circuitry.

In some embodiments of the examination unit, said electrical profile is characterized by application of an electrical current, comprising said electrical current profile, in a continuous or discontinuous manner, i.e., pulses, or any combination thereof.

In some embodiments of the examination unit, said at least one current electrode is configured for transmitting pulses of electrical current having a frequency in a range of about 1 kHz to about 10 MHz, with peak-to-peak voltage of about 0 to about 5 volt and with peak-to-peak current of about 40-70 micro Amper. The pulses may follow a sinusoidal pattern/wave or other pulse type. The frequency of the current is personalized and determined according to the subject's characteristics such as age, breast size (dimensions), volume, and tissue type.

The term “about” throughout the application should be understood as a deviation of ±20% from the nominal value. For example, if the nominal value is about 10 MHz, then it defines a range of between 8-12 MHz.

In some embodiments of the examination unit, the at least one attachable sheet is having flexibility for accommodating anatomical variety of different subjects and is at least partially made of a flexible and/or stretchable material; such that said examination unit is flexible to permit attaching to a majority of the selected first and second portions. In some embodiments of the examination unit, any one of said first or second layer or both are at least partially made of a flexible and/or stretchable material.

In some embodiments of the examination unit, said flexible material is selected from any one of: polyurethane, Polyester Nonwoven Tape and Non-Tackified acrylic-based adhesive, adhesive hydrogel, silica gel, or silicone adhesive.

In some embodiments, the examination unit further comprises a plurality of first pressure applicators associated with the at least one attachable sheet, each configured for controllably applying pressure on an area of the breast or the axilla associated therewith when said at least one attachable sheet is attached to the breast. Said first sensing spots are further configured for sensing from a skin portion associated therewith at least one of deformation profile, pressure profile or pressure change profile in response to controlled pressure application and generate respective data that is comprised in said first sensed data. The processing circuitry is further configured for controlling said first pressure applicators to execute a selected first pressure plan on the breast and/or axilla to thereby measure the response of the skin to the applied pressure.

In some embodiments of the examination unit, the first sensing spots comprise a plurality of first pressure sensors.

In some embodiments of the examination unit, each first sensing spot that comprises a first pressure sensor is associated with a respective first pressure applicator, such that the respective first pressure applicator is configured for applying pressure on at least the breast area associated with the respective first sensing spot.

In some embodiments of the examination unit, said selected first pressure plan comprises applying pressure simultaneously on selected mammary peripheral portions of the breast for a selected duration, followed by a coordinated relief of the pressure from said mammary peripheral portions. The processing circuitry is configured for operating said first sensing units to at least sense the temperature profile in response to said selected first pressure plan.

In some embodiments of the examination unit, said coordinated relief comprises relieving the pressure simultaneously from said mammary peripheral portions.

In some embodiments of the examination unit, said mammary peripheral portions form a continuum.

In some embodiments of the examination unit, said application of pressure on said selected peripheral portions results in a continuous pressure application along a force application line.

In some embodiments of the examination unit, said selected first pressure plan comprises: applying pressure with at least one first early pressure applicator and relieving the pressure application of said at least one first early pressure applicator; following the relief of pressure of the at least one first early pressure applicator, applying a pressure with at least one first subsequent pressure applicator and relieving the pressure application of said at least one first subsequent pressure applicator. The at least one first subsequent pressure applicator is arranged spatially consecutively to said at least one first early pressure applicator; and said processing circuitry is configured for operating said first sensing spots to at least sense the deformation profile, pressure profile or pressure change profile in response to said selected first pressure plan.

In some embodiments of the examination unit, said plurality of measuring electrodes comprises said at least one current electrode. Namely, some of the measuring electrodes can function also as current electrodes.

In some embodiments of the examination unit, each of said plurality of measuring electrodes functions also as a current electrode. Namely, each measuring electrode has two optional functions-(i) applying electrical current to at least a tissue portion associated with a respective said first sensing spot and (ii) measuring electrical profile, whether it is voltage, impedance or current.

In some embodiments of the examination unit, said plurality of measuring electrodes are clustered to a plurality of groups, each group comprises a plurality of measuring electrodes members. The processing circuitry is configured to operate said plurality of measuring electrodes to measure an electrical profile between each measuring electrode member of a group to any of other measuring electrode members at the same group, namely the electrical profile between each member and any other members is measured. Said electrical profile is selected from one or more of impedance, voltage and current. In a specific embodiment, the electrical profile is the impedance between each two measuring electrodes in the same group.

In some embodiments of the examination unit, some of the measuring electrodes are common to two or more groups. This overlap of measuring electrodes between different groups typically occurs in areas that statistically have the greatest chances to include an abnormality, such as a cancerous tissue. This may cause that such areas may undergo repetitive measurements between the same measuring electrodes or at least different measurements in different groups with a common measuring electrode.

In some embodiments of the examination unit, each measuring electrode is also a current electrode capable of applying an electric current to the tissue it is associated with. The processing circuitry is configured to control each measuring electrode such that for each measurement of electrical profile between each measuring electrode member of a group to any of other measuring electrode members, a selected profile of electric current is applied from one member participating in the measurement to another measurement participating in the measurement. Namely, in each measurement between a first member in the group and a second member in the group, an electric current is applied from the first member in the group towards the second member of the group.

In some embodiments of the examination unit, the electrical profile between each measuring electrode member of a group to any of other measuring electrode members of the group is measured without application of electrical current to measure the natural electrical profile of the breast, namely to map the natural electrical profile, such as voltage, of the breast. This is performed to identify tumors in the breast that transmit electrical signals for enhancing their proliferation rate. Such tumor cells contains greater charge than non-tumor cells and can be identify by this technique.

In some embodiments of the examination unit, the first sensing spots comprise said second sensing spots.

In some embodiments of the examination unit, said at least one attachable sheet comprises a first portion, a second portion and a third portion; the first portion and the second portion are shaped to fit over the shoulder of the subject, each from a different side of shoulder, so as to ensure a right placement of the at least one attachable sheet over the axilla and the breast of the subject; the third portion provides a degree of freedom with respect to the first and second portions to allow adjustment of the at least one attachable sheet to different sizes and shapes of breasts.

In some embodiments of the examination unit, each of the portions extends peripherally from a central portion of the at least one attachable sheet.

In some embodiments of the examination unit, the at least one attachable sheet defines a varying periphery length from a central point of the at least one attachable sheet to any point along the periphery of the at least one attachable sheet; wherein the peripheral length between two adjacent portions decrease to a certain minimum defining the border of the two adjacent portions.

In some embodiments of the examination unit, each of the three portions is a flap-like, or wing-like portion.

In some embodiments of the examination unit, the at least one attachable sheet comprises two similar sides, with all the sensing spots and electrodes, each side is intended to be attached to a different breast of the subject.

In some embodiments of the examination unit, said plurality of measuring electrodes are clustered to a plurality of circular groups, such that each measuring electrode in the circular group is at the same level as at least one other measuring electrode in the same group. The level should be understood as in a topographical map. Namely, the topographical height of such pairs of electrodes is the same with respect to the breast. The aim of such clustering is to allow a measurement of a deep breast tissue by allowing the current from one measuring electrode to pass through deeper tissues of the breast before reaching the other electrode. The term “circular” implies that the electrodes are arranged in a circular manner, which conforms with the shape of the breast and position these electrodes at about the same height or level with respect to the breast. However it is to be noted that these circular groups can include electrodes that are not arranged on a perfect circle and may deviate from the circular arrangement as long as they are positioned at about the same level or height with respect to the breast. The at least one processing circuitry is configured for operating the measuring electrodes to measure electrical profile between one measuring electrode of the circular group and at least one other measuring electrode in the group.

In some embodiments of the examination unit, the at least one processing circuitry is configured for operating the measuring electrodes to measure electrical profile between one measuring electrode of the circular group and an opposite measuring electrode of the group positioned at an opposite side of a circle defining the arrangement of the circular group. Namely, the measurement is performed between one electrode in the group and another measuring electrode that is positioned at the opposite side of the diameter of the circle that defines the group.

Yet another aspect of the present disclosure provides a system for performing examination of at least one breast of a subject and at least one axilla for identifying a tissue abnormality. The system comprising the examination unit of any one of the above-described embodiments or any combination thereof; an analyzing module configured for receiving and analyzing said at least first sensed data and identifying therein signatures indicative of abnormality in at least one of said selected first portion and said selected second portion of the subject and generating abnormality data based thereon; and an output module configured for outputting said abnormality data.

In some embodiments of the system, said analyzing module is further configured for receiving one or more user-related parameters indicative of at least one of: medical-related data of the subject; age; Body Mass Index (BMI); demographic data of the subject; genetic data of the subject; family history of the subject indicative of abnormalities of the breast, ovaries, and/or uterus; family history or personal history of the subject indicative of cancer and/or cancer-related pathologies; ethnicity; objective data of the subject, and; subjective data of the subject; and wherein said identifying is further based on said one or more user-related parameters.

In some embodiments of the system, the analyzing module is configured to receive bra size, breast volume or both bra size and breast volume. The analyzing module is configured to apply adjustment condition on the first sensed data based on the bra size or breast volume. The adjustment condition may be further dependent on the breast tissue density and fatty vs glandular components.

In some embodiments of the system, said analyzing comprises generating a sensing map of at least (i) an electrical profile, including any of impedance, permittivity, or conductance, or any combination thereof, and (ii) IR profile or thermal profile, based on said respective first sensed data.

In some embodiments of the system, said analyzing comprises to analyze said second sensing data to identify a heat signature of one or more anatomical landmarks for verifying a good placement of said attachable sheet, and if no heat signature is identified, the processing circuitry is configured to execute a misplacement alert.

In some embodiments of the system, said analyzing further comprises generating a sensing map of at least one of deformation profile, pressure profile or pressure change profile in response to controlled pressure application based on said respective first data.

In some embodiments of the system, said analyzing comprises comparing each of said respective first sensed data to a baseline data for identifying a variation from said baseline being indicative of an abnormality.

In some embodiments of the system, said baseline is selected from any one of: a respective sensed data of the contralateral breast and/or axilla, subjective tissue density of the examined breast, a pre-obtained sensed data of a subject having a certain degree of similarity to one or more said user-related parameters, other subjective and/or objective medical-related data adjusted to demographic data, such as age, BMI and ethnicity.

In some embodiments of the system, said analyzing comprises analysis of fat percentage of said first selected portion for determining said subjective tissue density. Fat percentage of the breast tissue is calculated according to impedance data recorded in the electrodes placed in the superior and inferior borders of the breast. Using this parameter, the breast density and structure are calculated and used in the analysis.

In some embodiments of the system, the analyzing module is configured to compare between first sensed data of two identical groups of first sensing spots, each measured in a different breast of the subject. In some embodiments, the comparison is made between each two similar groups of measuring electrodes, wherein each of the similar groups is measured on a different breast of the subject. The measurement can include a plurality of groups such that each group includes a plurality of measuring electrodes. The measurement of electrical profile is made between each two electrodes in the same group, namely, each group in one side of the breast has a corresponding group in the other side for comparison. This kind of measurement is performed identically on both breasts and therefore can be compared. If a significant variation between the two breasts is observed, it is included in the abnormality data. This can be also processed with the IR profile in the same suspected area to increase the confidence of the identified abnormality.

Yet another aspect of the present disclosure provides a method for performing examination of a breast of a subject, and optionally an axilla of the subject. The method comprising: attaching a plurality of measuring electrodes; attaching a plurality of first sensing spots and at least one current electrode to a first selected portion and, optionally a second selected portion, corresponding to at least one breast and one optionally axilla, respectively; wherein each first sensing spot for sensing from a tissue portion associated therewith either an (i) electrical profile by a respective measuring electrode from said plurality of measuring electrodes; (ii) IR radiation profile or thermal profile; or (iii) (i) electrical profile and (ii) IR radiation profile, and generate a respective first sensed data based thereon; wherein the at least one current electrode are configured for applying electrical current to at least a tissue portion associated with a respective said first sensing spot; wherein the method further comprising controlling said at least one current electrode to execute a selected electrical current profile; the method further comprises operating said first and second sensing spots and receiving each of said respective first and second sensed data; and transmitting each of said respective first sensed data.

In some embodiments of the method, said first selected portion comprises a majority of the breast area of said breast, defined as the area spanning between the clavicle bone and mammary fold.

In some embodiments of the method, said second selected portion comprises a majority of the axilla surface of said axilla.

In some embodiments of the method, said first selected portion and said second selected portion form a physical continuum being attachable to at least one said attachable sheet.

In some embodiments, the method further comprises attaching a plurality of second sensing spots to a first selected portion and, optionally a second selected portion, corresponding to at least one breast and one optionally axilla, respectively. Each second sensing spot is configured for sensing from a tissue portion associated therewith an IR radiation profile or thermal profile and generate a respective second sensed data based thereon.

In some embodiments of the method, at least a portion of the measuring electrodes constitute said at least one current electrode. Namely, the measuring electrode is capable of executing the desired current profile to be applied to the skin portion it is associated with and also measuring the electrical profile in response to the execution of said current profile.

In some embodiments of the method, said electrical profile is determined by any of impedance, conductivity, electrical potential of the tissue, and permittivity, or any combination thereof, of the at least said tissue portion being associated with a respective first sensing spot thereto.

In some embodiments of the method, said first sensing spots comprise a measuring electrode and a thermal sensor, whereby said electrode and said sensor are physically adjacent one to the other in each sensing spot. Namely, the thermal sensor and the measuring electrode are placed practically in the same place so that the data maps overlap.

In some embodiments of the method, each of the plurality of first sensing spots comprises a measuring electrode and a thermal sensor, and wherein said first sensing spot is configured for sensing both of (i) electrical profile; and (ii) IR radiation profile or thermal profile of the respective tissue portion associated therewith. It is to be noted that each sensing spot is defined by a certain area that includes at least one thermal sensor and at least one measuring electrode. By arranging the sensors in this manner, the generated maps of the electrical profile and the thermal profile can be correlated easily.

In some embodiments of the method, said plurality of first sensing spots are arranged in a first sensing array, each said sensing spot being positioned one with respect to the other along at least one first continuous line. The distance between the sensors may be equal or smaller than 5 mm in a constant-even distribution.

In some embodiments of the method, said at least one first continuous line is configured to accommodate the shape and dimensions of said first selected portion.

In some embodiments of the method, at least one of said continuous line defines a left-hand or right-hand spiral comprising said first sensing spots being arranged in a sequential and ordered manner and configured for permitting sensing of a majority of said selected first portion.

In some embodiments of the method, said continuous line forms a closed shape. In some embodiments, the method further comprises optically sensing the breast to identify an anatomical landmark of the breast and to generate optical sensed data; and identifying a misplacement of the examination unit based on the optical sensed data and outputting an alert if a misplacement is identified.

In some embodiments, the method further comprises performing optical sensing from portions proximal to the first or second sensing spots, from an attachable sheet that comprises them, optical parameters indicative of intensity profile of light of selected one or more wavelength ranges and generating optical sensed data based thereon. The method further comprises analyzing said optical sensed data and identifying at least one of: (1) inadequate level of adhesiveness of the attachable sheet and/or stability thereof throughout the examination process (including the first step of attachment of the examination unit); and (2) misplacement of the examination unit. The method further comprise outputting an alert if (1) or (2) or both is identified.

In some embodiments of the method, at least one of said continuous line defines an open-shape configuration configured for permitting sensing of a majority of said selected second portion.

In some embodiments of the method, said first sensing spots comprise said second sensing spots. Namely, the second sensing spots can also serve for performing the substantial measurements and not only for placement verification.

In some embodiments, the method further comprises arranging said second sensing sports in a second array, that can be part of the first array or different than the first array; each second sensing spot being positioned one with respect to the other along at least one second continuous line; wherein the method further comprising analyzing said second sensing data to identify a heat signature of one or more anatomical landmarks for verifying a good placement of said attachable sheet, and if no heat signature is identified, the method further comprising outputting a misplacement alert. The landmarks are typically one or both of the internal mammary artery and the lateral artery of each breast. It is to be noted that when the at least one attachable sheet is a single sheet, a good placement verification by one of these arteries may also indicate a good placement on the axilla portion. In some embodiments, it may further require a simple verification that the axilla portion of the attachable sheet is indeed attached to the skin of the subject.

In some embodiments of the method, said at least one second continuous line is disposed laterally to said first selected portion. For validation of correct placement of the breast unit: the lateral and medial borders of the device may contain a vertical line of heat sensors and optionally optic sensors, aimed at detecting the lateral thoracic artery, nipple, and the internal mammary artery, thus assuring that the device is in place.

In some embodiments of the method, said at least one second continuous line is medial to said first selected portion.

In some embodiments of the method, said electrical profile is characterized by application of an electrical current, comprising said electrical current profile, in a continuous or discontinuous manner, i.e., pulses, or any combination thereof.

In some embodiments of the method, said at least one current electrode is configured for transmitting pulses of electrical current having a frequency in a range of about 1 kHz to about 10 MHz, with peak-to-peak voltage of about 0 to about 5 volt and with peak-to-peak current of about 40-70 micro Amper.

In some embodiments, the method further comprising attaching a plurality of first pressure applicators, each configured for controllably applying pressure on an area of the breast or the axilla associated therewith; controlling said first pressure applicators to execute a selected first pressure plan on the breast and/or axilla, to thereby measure the response of the skin to the applied pressure; and sensing from a skin portion associated with the first sensing spots at least one of deformation profile, pressure profile or pressure change profile in response to controlled pressure application and generate respective data that is comprised in said first sensed data.

In some embodiments of the method, the first sensing spots comprise a plurality of first pressure sensors.

In some embodiments of the method, said selected first pressure plan comprises applying pressure simultaneously on selected mammary peripheral portions of the breast for a selected duration, followed by a coordinated relief of the pressure from said mammary peripheral portions; and operating said first sensing units to at least sense the temperature profile in response to said selected first pressure plan.

In some embodiments of the method, said coordinated relief comprises relieving the pressure simultaneously from said mammary peripheral portions.

In some embodiments of the method, said mammary peripheral portions form a continuum.

In some embodiments of the method, said application of pressure on said selected peripheral portions results in a continuous pressure application along a force application line.

In some embodiments of the method, said selected first pressure plan comprises:

applying pressure with at least one first early pressure applicator and relieving the pressure application of said at least one first early pressure applicator; following the relief of pressure of the at least one first early pressure applicator, applying a pressure with at least one first subsequent pressure applicator and relieving the pressure application of said at least one first subsequent pressure applicator; wherein said at least one first subsequent pressure applicator is arranged spatially consecutively to said at least one first early pressure applicator; and the method further comprising operating said first sensing units to at least sense the deformation profile, pressure profile or pressure change profile in response to said selected first pressure plan.

In some embodiments, the method further comprises analyzing said at least first sensed data and identifying therein signatures indicative of abnormality in at least one of said selected first portion and said selected second portion of the subject and generating abnormality data based thereon; and outputting said abnormality data.

In some embodiments, the method further comprises receiving one or more user-related parameters indicative of at least one of: medical-related data of the subject; demographic data of the subject; BMI; age; genetic data of the subject; family history of the subject indicative of abnormalities of the breast, ovaries, and/or uterus; family history or personal history of the subject indicative of cancer and/or cancer-related pathologies; ethnicity; objective data of the subject, and; subjective data of the subject; and wherein said identifying is further based on said one or more user-related parameters.

In some embodiments of the method, said analyzing further comprises generating a sensing map of at least one of deformation profile, pressure profile or pressure change profile in response to controlled pressure application based on said respective first data.

In some embodiments of the method, said analyzing comprises comparing each of said respective first sensed data to a baseline data for identifying a variation from said baseline being indicative of an abnormality.

In some embodiments of the method, said baseline is selected from any one of: a respective sensed data of the contralateral breast and/or axilla, subjective tissue density of the examined breast, a pre-obtained sensed data of a subject having a certain degree of similarity to one or more said user-related parameters, other subjective and/or objective medical-related data adjusted to demographic data, such as age, BMI and ethnicity.

In some embodiments, the method further comprises applying a selected current profile between one or more pairs of current electrodes that are positioned in opposite sides of the breast, namely distanced one from the other. Each member of said one or more pairs of electrodes is associated with a respective fat measuring electrode that is comprised in said plurality of measuring electrodes. The method further comprises measuring electrical profile by the respective fat measuring electrodes, wherein said electrical profile measured by the fat measuring electrodes is indicative of the fat percentage in the tissue. Typically, the electrical current and the voltage between the two fat measuring electrodes is sensed and analyzed to extract the fat percentage of the breast tissue. This measurement may be used in order to analyze the generated maps and to contribute to the conclusion whether an abnormality is identified in the breast or axilla or not.

In some embodiments of the method, said analyzing comprises analysis of fat percentage of said first selected portion for determining said subjective tissue density.

In some embodiments, the method further comprises receiving breast size data indicative of the bra size, breast volume or both bra size and breast volume. Said analyzing further comprises applying adjustment condition on the first sensed data based on the bra size or breast volume. The adjustment condition may be further dependent on the breast tissue density and fatty vs glandular components.

Yet another aspect of the present disclosure provides a breast unit for performing examination of a breast of a subject for identifying abnormality in said breast. The unit comprising a flexible attachable first sheet shaped for being attached, through one of its faces, to a desired portion of said breast. It is to be noted that the term “desired” throughout the application is interchangeable with the term “selected”. The first sheet comprises a plurality of first sensing units, each first sensing unit configured for sensing from a skin portion associated therewith at least one of (i) deformation profile, pressure profile or pressure change profile, and (ii) IR radiation profile or temperature profile of the respective skin portion and generate a respective first sensed data based thereon. It is to be noted that sensing from a skin portion including sensing parameters of behavior of the skin or through the skin, such as temperature. The breast unit further comprises a plurality of first pressure applicators associated with the first sheet, each configured for controllably applying pressure on an area of the breast associated therewith when said first sheet is attached to the breast. The breast unit further comprises a processing circuitry, i.e., a processor or a controller and one or more memories coupled to the processing circuitry and storing programming instructions for execution by the processing circuitry for:
(1) operating said first sensing units and receiving each of said respective first sensed data;
(2) controlling said first pressure applicators to execute a desired first pressure plan on the breast;
(3) transmitting each of said respective first sensed data from all the first sensing units to an external computing device for analyzing the first sensed data and identifying whether there is an abnormality or not.

It is to be noted that any combination of the described embodiments with respect to any aspect of the present disclosure is applicable. In other words, any aspect of the present disclosure can be defined by any combination of the described embodiments.

In some embodiments of the breast unit, said attachable first sheet comprises a skin facing face. The skin facing face comprises an adhesive layer for allowing attachment to said desired portion.

In some embodiments of the breast unit, said skin facing face comprises a removable cover covering said adhesive layer and configured for removal prior to said attachment.

In some embodiments of the breast unit, said desired portion comprises a majority of the breast area, including the nipple and areola.

In some embodiments of the breast unit, said desired portion comprises a majority of the breast area, excluding the nipple and areola.

In some embodiments of the breast unit, said first sheet has a shape of a closed shape, namely the shape of the sheet confining the nipple or including it in its area.

In some embodiments of the breast unit, said first sheet has a shape entirely encircling the nipple of the subject when being attached to said desired portion.

In some embodiments of the breast unit, said first sensing units comprise first pressure sensors. The pressure sensors may include any one of tactile sensor, piezoelectric sensor, magneto-resistive sensor, force sensitive resistor, etc.

In some embodiments of the breast unit, said first sensing units further comprise temperature sensors. The temperature sensors may include any one of IR sensor, resistance temperature detector (RTD), thermocouple, semiconductor IC, etc.

In some embodiments of the breast unit, each first sensing unit comprises a first pressure sensor and a temperature sensor, and wherein each first sensing unit is configured for sensing both the (i) deformation profile, pressure profile or pressure change profile, and (ii) IR radiation profile or temperature profile of the respective skin portion associated therewith.

In some embodiments of the breast unit, said first sheet comprises a first layer and a second layer, wherein the first layer comprises said first sensing units and the adhesive, and the second layer comprises said first pressure applicators.

In some embodiments of the breast unit, said second layer comprises said processing circuitry.

In some embodiments of the breast unit, said first sheet comprises said processing circuitry.

In some embodiments of the breast unit, each first sensing unit is associated with a respective first pressure applicator, such that the respective first pressure applicator is configured for applying pressure on a breast area associated with the respective first sensing unit.

In some embodiments, the first pressure applicators are arranged in a mesh. Each pressure sensor in the mesh is coupled to a plurality of own elastic wires or elements, namely elastic wires or elements that are only coupled to the specific pressure sensor, and the respective pressure applicator of each first pressure sensor is configured to apply pressure that results in a deformation of the elasticity of the elastic wires coupled with the respective pressure sensor. This configuration allows application of pressure on an isolated breast portion and the measurement of a pressure-indicative parameter of the isolated breast portion in response to the application of pressure thereon.

In some embodiments of the breast unit, said desired first pressure plan comprises applying pressure simultaneously on selected mammary peripheral portions of the breast, or of the sheet that results in an application of pressure on the breast, for a selected duration, followed by a coordinated relief of the pressure from said mammary peripheral portions. The peripheral pressure application results in a reduction of blood flow toward internal regions of the breast tissue that are confined by the selected peripheral portions, causing these internal regions to cool. Following pressure relief, the sensing units sense the temperature profile of each skin portion, which may exhibit a signature that may be indicative of an abnormality in said skin portion. The processing circuitry is further configured for operating said first sensing units to at least sense the temperature profile in response to said desired first pressure plan.

In some embodiments of the breast unit, said coordinated relief comprises relieving the pressure simultaneously from said mammary peripheral portions.

In some embodiments of the breast unit, said mammary peripheral portions form a continuum, e.g., a spatial continuum. It is to be noted that the continuum is manifested by given pressure that is applied along a peripheral circle encircling the nipple.

In some embodiments of the breast unit, said application of pressure on said selected peripheral portions results in a continuous pressure application along a force application line, which may not be equal at any point of said line but at least greater than a passive state when no application of force is applied. It is to be noted that the application line is not tangible, and only aims to define the geometric shape of the application of the pressure.

In some embodiments of the breast unit, said desired first pressure plan comprises:
(a) applying pressure with at least one first early (or primary) pressure applicator and relieving the pressure application of said at least one first early pressure applicator;
(b) following the relief of pressure of the at least one first early pressure applicator, applying a pressure with at least one first subsequent pressure applicator and relieving the pressure application of said at least one first subsequent pressure applicator.

Said at least one first subsequent pressure applicator is arranged spatially consecutively to said at least one first early pressure applicator, thereby resulting in a measurement of pressure change or deformation profile of consecutive portions of the breast allowing to create a consecutive map of pressure change or deformation profile behavior of the breast in response to application of pressure. The processing circuitry is further configured for operating said first sensing units to at least sense the deformation profile, pressure profile or pressure change profile in response to said desired first pressure plan.

In some embodiments of the breast unit, said processing circuitry is electrically connected to each of said first sensing units to thereby control their operation and receive said respective first sensed data.

In some embodiments of the breast unit, said processing circuitry is electrically connected to each of said first pressure applicators to thereby control their operation.

In some embodiments, the breast unit is disposable.

In some embodiments of the breast unit, said sheet is formed, at least partially, of any one of silicon, plastic TPU or PDMS.

Yet another aspect of the present disclosure provides a kit for performing examination of one or more body parts of a subject for identifying abnormality in said one or more body parts. The kit comprising the breast unit of any one of the above-described embodiments or any combination thereof. The kit further comprises an axillary unit that comprises (i) an attachable second sheet shaped for being attached to a desired portion of an armpit of the subject, said second sheet comprising a plurality of second sensing units, each second sensing unit configured for sensing from a skin portion associated therewith a deformation profile, pressure profile or pressure change profile, and generate a respective second sensed data based thereon; (ii) a plurality of second pressure applicators associated with said second sheet, each configured for controllably applying pressure on an area of the armpit associated therewith when said second sheet is attached to the armpit; and (iii) a transmitting unit for transmitting each of said respective second sensed data.

In some embodiments of the kit, the processing circuitry is configured for:
(1) operating said second sensing units;
(2) controlling said second pressure applicators to execute a desired second pressure plan on the armpit.

In some embodiments of the kit, said processing circuitry is further configured for receiving each of said respective second sensed data and transmitting each of said respective second sensed data.

In some embodiments of the kit, said attachable second sheet comprises a second skin facing face, said second skin facing face comprises an adhesive layer for allowing attachment to said desired portion.

In some embodiments of the kit, said second skin facing face comprises a second removable cover covering said adhesive layer and configured for removal prior to said attachment.

In some embodiments of the kit, said second sensing units comprise second pressure sensors configured for sensing the deformation profile, pressure profile or pressure change profile of the respective skin portion associated therewith.

In some embodiments of the kit, said second sheet comprises a first layer and a second layer, and wherein the first layer comprises said second sensing units and the second layer comprises said second pressure applicators.

In some embodiments of the kit, the second layer of said second sheet comprises said transmitting unit.

In some embodiments of the kit, each second sensing unit is associated with a respective second pressure applicator, such that the respective second pressure applicator is configured for applying pressure on an axillary area associated with the respective second sensing unit.

In some embodiments, the second pressure applicators are arranged in a mesh. Each pressure sensor in the mesh is coupled to a plurality of own elastic wires, namely elastic wires or elements that are only coupled to the specific pressure sensor, and the respective pressure applicator of each second pressure sensor is configured to apply pressure that results in a deformation of the elasticity of the elastic wires coupled with the respective pressure sensor. This configuration allows application of pressure on an isolated breast portion and the measurement of a pressure-indicative parameter of the isolated breast portion in response to the application of pressure thereon.

In some embodiments of the kit, said desired second pressure plan comprises:
applying pressure with at least one second early pressure applicator and relieving the pressure application of said at least one second early pressure applicator; following the relief of pressure of the at least one second early pressure applicator, applying pressure with at least one second subsequent pressure applicator and relieving the pressure application of said at least one second subsequent pressure applicator; and wherein said at least one second subsequent pressure applicator is arranged spatially consecutively to said at least one second early pressure applicator. The processing circuitry is further configured for operating said second sensing units to at least sense the deformation profile, pressure profile or pressure change profile in response to said desired second pressure plan.

In some embodiments of the kit, said transmitting unit is configured for transmitting said second sensed data to one of the processing circuitry and an external computing unit.

In some embodiments of the kit, said axillary unit is disposable.

In some embodiments of the kit, said second sheet is formed, at least partially, of any one of silicon, plastic TPU or PDMS.

Yet another aspect of the present disclosure provides a system for performing examination of a breast of a subject and identifying abnormality in said breast. The system comprising the breast unit or the kit of any one of the above-described embodiments or any combination thereof; and an analyzing module configured for receiving and analyzing said respective first sensed data, the second sensed data, or the first and second data, and identifying therein signatures indicative of abnormality in the breast or the armpit of the subject.

In some embodiments of the system, said analyzing module is further configured for receiving one or more user-related parameters indicative of at least one of medical-related data of the subject, demographic data of the subject, genetic data of the subject, family history indicative of abnormalities of the breast, ethnicity, objective data of the subject, such as age, subjective data of the subject, such as clinical symptoms and the like, and wherein said identifying is further based on said one or more user-related parameters.

In some embodiments of the system, said analyzing comprises generating a map of (i) deformation profile, pressure profile or pressure change profile, or (ii) IR radiation profile or temperature profile based on each of the respective first sensed data, second sensed data, or the first and second sensed data.

In some embodiments of the system, said analyzing comprises comparing each of said respective first sensed data, second sensed data, or the first and the second sensed data to a baseline data for identifying a variation from said baseline being indicative of an abnormality.

In some embodiments of the system, said baseline is selected from any one of a respective sensed data of a different skin portion, a pre-obtained sensed data of a subject having a certain degree of similarity to one or more user-related parameters, other women's data adjusted to age, ethnicity and breast density, or a respective sensed data of a different breast of the subject.

Yet another aspect of the present disclosure provides a method for performing examination of a breast of a subject for identifying abnormality in said breast. The method comprising attaching a plurality of first sensing units to said breast, each first sensing unit configured for sensing from a skin portion associated therewith at least one of (i) deformation profile, pressure profile or pressure change profile, and (ii) IR radiation profile or temperature profile of the respective skin portion and generate a respective first sensed data based thereon; attaching a plurality of first pressure applicators to said breast, each configured for controllably applying pressure on an area of the breast associated therewith; operating said first sensing units and receiving each of said respective first sensed data; controlling said first pressure applicators to execute a desired first pressure plan on the breast; transmitting each of said respective first sensed data from all the first sensing units to an external computing device for analyzing the first sensed data and identifying whether there may be an abnormality or not.

In some embodiments of the method, said first sensing units comprise first pressure sensors. The pressure sensors may include any one of tactile sensor, piezoelectric sensor, magneto-resistive sensor, force sensitive resistor.

In some embodiments of the method, said first sensing units further comprise temperature sensors. The temperature sensors may include any one of IR sensor, resistance temperature detector (RTD), thermocouple, semiconductor IC, etc.

In some embodiments of the method, each first sensing unit comprises a first pressure sensor and a temperature sensor, and wherein each first sensing unit is configured for sensing both the (i) deformation profile, pressure profile or pressure change profile, and (ii) the IR radiation profile or temperature profile of the respective skin portion associated therewith.

In some embodiments of the method, said desired first pressure plan comprises applying pressure simultaneously on selected mammary peripheral portions of the breast for a selected duration, followed by a coordinated relief of the pressure from said mammary peripheral portions. The peripheral pressure application results in a reduction of blood flow toward internal regions of the breast tissue that are confined by the selected peripheral portions, causing these internal regions to cool. Following pressure relief, the sensing units sense the temperature profile of each skin portion, which may exhibit a signature that may be indicative of abnormality in said skin portion. The method further comprises operating said first sensing units to at least sense the temperature profile in response to said desired first pressure plan.

In some embodiments of the method, said coordinated relief comprises relieving the pressure simultaneously from said mammary peripheral portions.

In some embodiments of the method, said mammary peripheral portions form a continuum, e.g., a spatial continuum. It is to be noted that the continuum is manifested by given pressure that is applied along a peripheral circle encircling the nipple.

In some embodiments of the method, said application of pressure on said selected peripheral portions results in a continuous pressure application along a force application line, which may not be equal at any point of said line but at least greater than a passive state when no application of force is applied. It is to be noted that the application line is not tangible, and only aims to define the geometric shape of the application of the pressure.

In some embodiments of the method, said desired first pressure plan comprises:
applying pressure with at least one first early/primary pressure applicator and relieving the pressure application of said at least one first early pressure applicator; following the relief of pressure of the at least one first early pressure applicator, applying a pressure with at least one first subsequent pressure applicator and relieving the pressure application of said at least one first subsequent pressure applicator; wherein said at least one first subsequent pressure applicator is arranged spatially consecutively to said at least one first early pressure applicator, thereby resulting in a measurement of pressure change or deformation profile of consecutive portions of the breast allowing to create a consecutive map of pressure change or deformation profile behavior of the breast in response to application of pressure. The method further operating said first sensing units to at least sense the deformation profile, pressure profile or pressure change profile in response to said desired first pressure plan.

In some embodiments, the method further comprises attaching a plurality of second sensing units to an armpit of the subject, each second sensing unit configured for sensing from a skin portion associated therewith a deformation profile, pressure profile or pressure change profile, and generate a respective second sensed data based thereon; attaching a plurality of second pressure applicators to said armpit, each configured for controllably applying pressure on an area of the armpit associated therewith; operating said second sensing units; controlling said second pressure applicators to execute a desired second pressure plan on the armpit; and transmitting each of said respective second sensed data.

In some embodiments of the method, said second sensing units comprise second pressure sensors configured for sensing the deformation profile, pressure profile or pressure change profile of the respective skin portion associated therewith.

In some embodiments of the method, each second sensing unit is associated with a respective second pressure applicator, such that the respective second pressure applicator is configured for applying pressure on an axillary area associated with the respective second sensing unit.

In some embodiments of the method, said desired second pressure plan comprises:
applying pressure with at least one second early pressure applicator and relieving the pressure application of said at least one second early pressure applicator; following the relief of pressure of the at least one second early pressure applicator, applying pressure with at least one second subsequent pressure applicator and relieving the pressure application of said at least one second subsequent pressure applicator. Wherein said at least one second subsequent pressure applicator is arranged spatially consecutively to said at least one second early pressure applicator. The method further comprises operating said second sensing units to at least sense the deformation profile, pressure profile or pressure change profile in response to said desired second pressure plan.

In some embodiments, the method further comprises analyzing said respective first sensed data, second sensed data, or the first and the second sensed data, and identifying therein signatures indicative of abnormality in the breast or the armpit of the subject.

In some embodiments, the method further comprises receiving one or more user-related parameters indicative of at least one of: medical-related data of the subject, demographic data of the subject, genetic data of the subject, family history indicative of abnormalities of the breast, ethnicity, objective data of the subject, such as age, subjective data of the subject, such as clinical symptoms and the like, wherein said identifying is further based on said one or more user-related parameters.

In some embodiments of the method, said analyzing comprises generating a map of (i) deformation profile, pressure profile or pressure change profile, or (ii) IR radiation profile or temperature profile based on each of the respective first sensed data, the second sensed data, or the first and second sensed data.

In some embodiments of the method, said analyzing comprises comparing each of said respective first sensed data, second sensed data, or the first and second sensed data to a baseline data for identifying a variation from said baseline being indicative of an abnormality.

In some embodiments of the method, said baseline is selected from any one of: a respective sensed data of a different skin portion, a pre-obtained sensed data of a subject having a certain degree of similarity to one or more user-related parameters, other women's data adjusted to age, ethnicity and breast density, or a respective sensed data of a different breast of the subject.

In some embodiments of the method, said plurality of measuring electrodes comprises said at least one current electrode. Namely, some of the measuring electrodes can function also as current electrodes.

In some embodiments of the method, each of said plurality of measuring electrodes functions also as a current electrode. Namely, each measuring electrode has two optional functions—(i) applying electrical current to at least a tissue portion associated with a respective said first sensing spot and (ii) measuring electrical profile, whether it is voltage, impedance or current.

In some embodiments of the method, said plurality of measuring electrodes are clustered to a plurality of groups, each group comprises a plurality of measuring electrodes members; wherein the method further comprises measuring an electrical profile between each measuring electrode member of a group to any of other measuring electrode members at the same group, namely the electrical profile between each member and any other members is measured; wherein said electrical profile is selected from one or more of impedance, voltage and current. In a specific embodiment, the electrical profile is the impedance between each two measuring electrodes in the same group.

In some embodiments of the method, each measuring electrode is also a current electrode capable of applying an electric current to the tissue it is associated with; wherein for each measurement of electrical profile between each measuring electrode member of a group to any of other measuring electrode members, a selected profile of electric current is applied from one member participating in the measurement to another measurement participating in the measurement. Namely, in each measurement between a first member in the group and a second member in the group, an electric current is applied from the first member in the group towards the second member of the group.

In some embodiments of the method, the electrical profile between each measuring electrode member of a group to any of other measuring electrode members of the group is measured without application of electrical current to measure the natural electrical profile of the breast, namely to map the natural electrical profile, such as voltage, of the breast.

In some embodiments of the method, the first sensing spots comprise said second sensing spots.

In some embodiments of the method, said plurality of measuring electrodes are clustered to a plurality of circular groups, such that each measuring electrode in the circular group is at the same level as at least one other measuring electrode in the same group. The level should be understood as in a topographical map. Namely, the topographical height of such pairs of electrodes is the same with respect to the breast. The aim of such clustering is to allow a measurement of a deep breast tissue by allowing the current from one measuring electrode to pass through deeper tissues of the breast before reaching the other electrode. The term “circular” implies that the electrodes are arranged in a circular manner, which conforms with the shape of the breast and position these electrodes at about the same height or level with respect to the breast. However it is to be noted that these circular groups can include electrodes that are not arranged on a perfect circle and may deviate from the circular arrangement as long as they are positioned at about the same level or height with respect to the breast). The method further comprises measuring electrical profile between one measuring electrode of the circular group and at least one other measuring electrode in the group.

In some embodiments, the method further comprises measuring electrical profile between one measuring electrode of the circular group and an opposite measuring electrode of the group positioned at an opposite side of a circle defining the arrangement of the circular group. Namely, the measurement is performed between one electrode in the group and another measuring electrode that is positioned at the opposite side of the diameter of the circle that defines the group.

### EMBODIMENTS

The following are optional embodiments and combinations thereof in accordance with aspects of the present disclosure:
1. An examination unit for performing examination of a breast of a subject, and optionally an axilla of the subject, for identifying a tissue abnormality, comprising:
   at least one attachable sheet configured for being attached to a first selected portion and, optionally a second selected portion, corresponding to at least one breast and one optionally axilla, respectively, wherein said attachable sheet comprises:
      a plurality of measuring electrodes;
      a plurality of first sensing spots, each sensing spot configured for sensing from a tissue portion associated therewith either (i) electrical profile by a respective measuring electrode from said plurality of measuring electrodes; (ii) IR radiation profile or thermal profile; or (iii) both (i) and (ii), and generate a respective first sensed data based thereon;
      optionally, a plurality of second sensing spots, each second sensing spot configured for sensing from a tissue portion associated therewith an IR radiation profile or thermal profile and generate a respective second sensed data based thereon (namely, it should be understood that the broadest definition of the examination unit does not include the plurality of second sensing spots and in a more specific, optional, definition it includes the plurality of second sensing spots);
      at least one current electrode configured for applying electrical current to at least a tissue portion associated with a respective said first sensing spot;
   the examination unit further comprising a processing circuitry; one or more memories coupled to the processing circuitry; and storing programming instructions for execution by the processing circuitry for:
      (i) operating said first and second sensing spots and receiving each of said respective first and second sensed data;
      (ii) controlling said at least one current electrode to execute a selected electrical current profile; and
      (iii) transmitting each of said respective first and second sensed data.
2. The examination unit of embodiment 1, wherein said at least one attachable sheet comprises a skin facing face, said skin facing face comprises an adhesive layer for allowing attachment to said first and second selected portions.
3. The examination unit of embodiment 2, wherein said skin facing face comprises a removable cover covering said adhesive layer and configured for removal prior to said attachment.
4. The examination unit of any one of embodiments 1-3, wherein said at least one attachable sheet has a shape of a closed shape.
5. The examination unit of any one of embodiments 1-4, wherein said first selected portion comprises a majority of the breast area of said breast.
6. The examination unit of any one of embodiments 1-5, wherein said second selected portion comprises a majority of the axilla surface of said axilla.
7. The examination unit of any one of embodiments 1-6, wherein said first selected portion and said second selected portion form a physical continuum being attachable to at least one said attachable sheet.
8. The examination unit of any one of embodiments 1-7, wherein said at least one attachable sheet comprises a first attachable sheet attachable to said first selected portion and a second attachable sheet attachable to said second selected portion, wherein said first and second attachable sheets are physically discrete.
9. The examination unit of any one of embodiments 1-8, wherein at least a portion of the measuring electrodes constitute said at least one current electrode.
10. The examination unit of any one of embodiments 1-9, comprising a proportion of (1:1) of measuring electrodes and thermal sensors.
11. The examination unit of any one of embodiments 1-10, wherein said electrical profile is determined by any of impedance, conductivity, and permittivity, or any combination thereof, of the at least said tissue portion being associated with a respective first sensing spot thereto.
12. The examination unit of any one of embodiments 1-11, wherein said first sensing spots comprise a measuring electrode and a thermal sensor, whereby said electrode and said sensor are physically adjacent one to the other in each sensing spot.
13. The examination unit of any one of embodiments 1-12, wherein each of the plurality of first sensing spots comprises a measuring electrode and a thermal sensor, and wherein said first sensing spot is configured for sensing both of (i) electrical profile; and (ii) IR radiation profile or thermal profile of the respective tissue portion associated therewith.
14. The examination unit of any one of embodiments 1-13, wherein said at least one attachable sheet comprises a first sensing array having a plurality of said first sensing spots; each said sensing spot being positioned one with respect to the other along at least one first continuous line.
15. The examination unit of embodiment 14, wherein said at least one first continuous line is configured to accommodate the shape and dimensions of said first selected portion.
16. The examination unit of embodiment 15, wherein at least one of said continuous line defines a left-hand or right-hand spiral comprising said first sensing spots being arranged in a sequential and ordered manner and configured for permitting sensing of a majority of said selected first portion.
17. The examination unit of embodiment 16, wherein said continuous line forms a closed shape.
18. The examination unit of embodiment 17, wherein said first sensing array comprises a plurality of said closed shapes arranged as concentric circles.
19. The examination unit of embodiment 18, wherein the distance between each concentric circle is equal.
20. The examination unit of any one of embodiments 1-19, comprising one or more optical sensors disposed on different portions of a periphery of the at least one attachable sheet and configured for sensing optical parameters indicative of light intensity of one or more selected wavelength ranges and generating optical sensed data based thereon;
   wherein the processing circuitry is configured for analyzing said optical sensed data to identify insufficient level of adhesiveness or adherence of said at least one attachable sheet to the skin of the subject and outputting an alert if said insufficient level of adhesiveness is identified.
21. The examination unit of embodiment 13, wherein at least one of said continuous line defines an open-shape configuration configured for permitting sensing of a majority of said selected second portion.
22. The examination unit of any one of embodiments 1-21, wherein said first sensing spots comprise said second sensing spots.
23. The examination unit of any one of embodiments 1-22, comprising a second sensing array having a plurality of said second sensing spots; each second sensing spot being positioned one with respect to the other along at least one second continuous line, wherein the processing circuitry is further configured to analyze said second sensing data and to identify a heat signature of one or more anatomical landmarks for verifying a good placement of said attachable sheet, and if no heat signature is identified, the processing circuitry is configured to execute a misplacement alert.
24. The examination unit of embodiment 23, wherein said at least one second continuous line is lateral to said first selected portion.
25. The examination unit of any one of embodiments 23-24, wherein said at least one second continuous line is medial to said first selected portion.
26. The examination unit of any one of embodiments 1-25, wherein said attachable sheet comprises a first layer and a second layer, and wherein the first layer comprises said first and second sensing arrays and at least one current electrode; and the second layer comprises said processing circuitry.
27. The examination unit of any one of embodiments 1-26, wherein said electrical profile is characterized by application of an electrical current, comprising said electrical current profile, in a continuous or discontinuous manner, or any combination thereof.
28. The examination unit of any one of embodiments 1-27, wherein said at least one current electrode is configured for transmitting pulses of electrical current having a frequency in a range of about 1 kHz to about 10 MHz, with peak-to-peak voltage of about 0 to about 5 volt and with peak-to-peak current of about 40-70 micro Amper.
29. The examination unit of any one of embodiments 1-28, wherein the at least one attachable sheet is having flexibility for accommodating anatomical variety of different subjects and is at least partially made of a flexible and/or stretchable material; such that said examination unit is flexible to permit attaching to a majority of the selected first and second portions.
30. The examination unit of any one of embodiments 1-29, wherein any one of said first or second layer or both are at least partially made of a flexible and/or stretchable material.
31. The examination unit of any one of embodiments 29-30, wherein said flexible material is selected from any one of: polyurethane, Polyester Nonwoven Tape and Non-Tackified acrylic-based adhesive, adhesive hydrogel, silica gel, or silicone adhesive.
32. The examination unit of any one of embodiments 1-31, comprising a plurality of first pressure applicators associated with the at least one attachable sheet, each configured for controllably applying pressure on an area of the breast or the axilla associated therewith when said at least one attachable sheet is attached to the breast;
   wherein said first sensing spots are further configured for sensing from a skin portion associated therewith at least one of deformation profile, pressure profile or pressure change profile in response to controlled pressure application and generate respective data that is comprised in said first sensed data; and
   wherein the processing circuitry is further configured for controlling said first pressure applicators to execute a selected first pressure plan on the breast and/or axilla.
33. The examination unit of any one of embodiments 1-32, wherein the first sensing spots comprise a plurality of first pressure sensors.
34. The examination unit of any one of embodiments 1-33, wherein each first sensing spot that comprises a first pressure sensor is associated with a respective first pressure applicator, such that the respective first pressure applicator is configured for applying pressure on at least the breast area associated with the respective first sensing spot.
35. The examination unit of any one of embodiments 1-34, wherein said selected first pressure plan comprises applying pressure simultaneously on selected mammary peripheral portions of the breast for a selected duration, followed by a coordinated relief of the pressure from said mammary peripheral portions; and
   wherein said processing circuitry is configured for operating said first sensing units to at least sense the temperature profile in response to said selected first pressure plan.
36. The examination unit of embodiment 35, wherein said coordinated relief comprises relieving the pressure simultaneously from said mammary peripheral portions.
37. The examination unit of embodiment 35 or 36, wherein said mammary peripheral portions form a continuum.
38. The examination unit of any one of embodiments 35-37, wherein said application of pressure on said selected peripheral portions results in a continuous pressure application along a force application line.
39. The examination unit of any one of embodiments 31-38, wherein said selected first pressure plan comprises:
   applying pressure with at least one first early pressure applicator and relieving the pressure application of said at least one first early pressure applicator,
   following the relief of pressure of the at least one first early pressure applicator, applying a pressure with at least one first subsequent pressure applicator and relieving the pressure application of said at least one first subsequent pressure applicator,
   wherein said at least one first subsequent pressure applicator is arranged spatially consecutively to said at least one first early pressure applicator; and
   wherein said processing circuitry is configured for operating said first sensing spots to at least sense the deformation profile, pressure profile or pressure change profile in response to said selected first pressure plan.
40. The examination unit of any one of embodiments 1-39, wherein said plurality of measuring electrodes comprises said at least one current electrode.
41. The examination unit of any one of embodiments 1-40, wherein each of said plurality of measuring electrodes functions also as a current electrode.
42. The examination unit of any one of embodiments 1-41, wherein said plurality of measuring electrodes are clustered to a plurality of groups, each group comprises a plurality of measuring electrodes members; wherein said processing circuitry is configured to operate said plurality of measuring electrodes to measure an electrical profile between each measuring electrode member of a group to any of other measuring electrode members at the same group; wherein said electrical profile is selected from one or more of impedance, voltage and current.
43. The examination unit of embodiment 42, wherein each measuring electrode is also a current electrode capable of applying an electric current to the tissue it is associated with; wherein the processing circuitry is configured to control each measuring electrode such that for each measurement of electrical profile between each measuring electrode member of a group to any of other measuring electrode members, a selected profile of electric current is applied from one member participating in the measurement to another measurement participating in the measurement.
44. The examination unit of embodiment 42, wherein the electrical profile between each measuring electrode member of a group to any of other measuring electrode members of the group is measured without application of electrical current to measure the natural electrical profile of the breast.
45. The examination unit of any one of embodiments 1-44, wherein the first sensing spots comprise said second sensing spots.
46. The examination unit of any one of embodiments 1-45, wherein said at least one attachable sheet comprises a first portion, a second portion and a third portion; the first portion and the second portion are shaped to fit over the shoulder of the subject, each from a different side of shoulder, so as to ensure a right placement of the at least one attachable sheet over the axilla and the breast of the subject; the third portion provides a degree of freedom with respect to the first and second portions to allow adjustment of the at least one attachable sheet to different sizes and shapes of breasts.
47. The examination unit of embodiment 46, wherein each of the portions extends peripherally from a central portion of the at least one attachable sheet.
48. The examination unit of embodiment 47, wherein the at least one attachable sheet defines a varying periphery length from a central point of the at least one attachable sheet to any point along the periphery of the at least one attachable sheet; wherein the peripheral length between two adjacent portions decrease to a certain minimum defining the border of the two adjacent portions.
49. The examination unit of any one of embodiments 46-48, wherein each of the three portions is a flap-like portion.
50. The examination unit of any one of embodiments 1-49, wherein the at least one attachable sheet comprises two similar sides, each side is intended to be attached to a different breast of the subject.
51. The examination unit of any one of embodiments 1-50, wherein said plurality of measuring electrodes are clustered to a plurality of circular groups, such that each measuring electrode in the circular group is at the same level as at least one other measuring electrode in the same group; wherein the at least one processing circuitry is configured for operating the measuring electrodes to measure electrical profile between one measuring electrode of the circular group and at least one other measuring electrode in the group.
52. The examination unit of embodiment 51, wherein the at least one processing circuitry is configured for operating the measuring electrodes to measure electrical profile between one measuring electrode of the circular group and an opposite measuring electrode of the group positioned at an opposite side of a circle defining the arrangement of the circular group.
53. A system for performing examination of at least one breast of a subject and at least one axilla for identifying a tissue abnormality, comprising:
   the examination unit of any one of embodiments 1-52
   an analyzing module configured for receiving and analyzing said at least first sensed data and identifying therein signatures indicative of abnormality in at least one of said selected first portion and said selected second portion of the subject and generating abnormality data based thereon;
   an output module configured for outputting said abnormality data.
54. The system of embodiment 53, wherein said analyzing module is further configured for receiving one or more user-related parameters indicative of at least one of: medical-related data of the subject; age; BMI, demographic data of the subject; genetic data of the subject; family history or personal history of the subject indicative of abnormalities of the breast, ovaries, and/or uterus; family history of the subject indicative of cancer and/or cancer-related pathologies; ethnicity; objective data of the subject, and; subjective data of the subject; and wherein said identifying is further based on said one or more user-related parameters.
55. The system of any one of embodiments 53-54, wherein said analyzing comprises generating a sensing map of at least (i) an electrical profile, including any of impedance, permittivity, electrical potential of the tissue, or conductance, or any combination thereof, and (ii) IR profile or thermal profile, based on said respective first sensed data.
56. The system of any one of embodiments 53-55, wherein said analyzing comprises to analyze said second sensing data to identify a heat signature of one or more anatomical landmarks for verifying a good placement of said attachable sheet, and if no heat signature is identified, the processing circuitry is configured to execute a misplacement alert.
57. The system of embodiment 56, wherein said analyzing further comprises generating a sensing map of at least one of deformation profile, pressure profile or pressure change profile in response to controlled pressure application based on said respective first data.
58. The system of any one of embodiments 53-57, wherein said analyzing comprises comparing each of said respective first sensed data to a baseline data for identifying a variation from said baseline being indicative of an abnormality.
59. The system of embodiment 58, wherein said baseline is selected from any one of: a respective sensed data of the contralateral breast and/or axilla, subjective tissue density of the examined breast, a pre-obtained sensed data of a subject having a certain degree of similarity to one or more said user-related parameters, other subjective and/or objective medical-related data adjusted to demographic data, such as age and ethnicity.
60. The system of any one of embodiments 53-59, wherein said analyzing comprises analysis of fat percentage of said first selected portion for determining said subjective tissue density.
61. The system of any one of embodiments 53-60, wherein the analyzing module is configured to compare between first sensed data of two identical groups of first sensing spots, each measured in a different breast of the subject.
62. A method for performing examination of a breast of a subject, and optionally an axilla of the subject, comprising:
   attaching a plurality of measuring electrodes;
   attaching a plurality of first sensing spots, at least one current electrode, and optional a plurality of second sensing spots (namely, it should be understood that the broadest definition of the method does not include the plurality of second sensing spots and in a more specific, optional, definition it includes the plurality of second sensing spots) to a first selected portion and, optionally a second selected portion, corresponding to at least one breast and one optionally axilla, respectively;
   wherein each first sensing spot for sensing from a tissue portion associated therewith an (i) electrical profile by a respective measuring electrode from said plurality of measuring electrodes; and (ii) IR radiation profile or thermal profile, and generate a respective first sensed data based thereon;
   wherein each second sensing spot configured for sensing from a tissue portion associated therewith an IR radiation profile or thermal profile and generate a respective second sensed data based thereon wherein the at least one current electrode are configured for applying electrical current to at least a tissue portion associated with a respective said first sensing spot;
   wherein the method further comprising controlling said at least one current electrode to execute a selected electrical current profile;
   operating said first and second sensing spots and receiving each of said respective first and second sensed data; and
   transmitting each of said respective first sensed data.
63. The method of embodiment 62, wherein said first selected portion comprises a majority of the breast area of said breast.
64. The method of embodiment 62 or 63, wherein said second selected portion comprises a majority of the axilla surface of said axilla.
65. The method of any one of embodiments 62-64, wherein said first selected portion and said second selected portion form a physical continuum being attachable to at least one said attachable sheet.
66. The method of any one of embodiments 62-65, wherein at least a portion of the measuring electrodes constitute said at least one current electrode.
67. The method of any one of embodiments 62-66, wherein said electrical profile is determined by any of impedance, electrical potential of the tissue, conductivity, and permittivity, or any combination thereof, of the at least said tissue portion being associated with a respective first sensing spot thereto.
68. The method of any one of embodiments 62-67, wherein said first sensing spots comprise a measuring electrode and a thermal sensor, whereby said electrode and said sensor are physically adjacent one to the other in each sensing spot.
69. The method of any one of embodiments 62-68, wherein each of the plurality of first sensing spots comprises a measuring electrode and a thermal sensor, and wherein said first sensing spot is configured for sensing both of (i) electrical profile; and (ii) IR radiation profile or thermal profile of the respective tissue portion associated therewith.
70. The method of any one of embodiments 62-69, wherein said plurality of first sensing spots are arranged in a first sensing array, each said sensing spot being positioned one with respect to the other along at least one first continuous line.
71. The method of embodiment 70, wherein said at least one first continuous line is configured to accommodate the shape and dimensions of said first selected portion.
72. The method of embodiment 71, wherein at least one of said continuous line defines a left-hand or right-hand spiral comprising said first sensing spots being arranged in a sequential and ordered manner and configured for permitting sensing of a majority of said selected first portion.
73. The method of embodiment 71, wherein said continuous line forms a closed shape.
74. The method of any one of embodiments 62-73, comprising optically sensing the breast to identify an anatomical landmark of the breast and to generate optical sensed data; and
   identifying a misplacement of the examination unit based on the optical sensed data and outputting an alert if a misplacement is identified.
75. The method of embodiment 70, wherein at least one of said continuous line defines an open-shape configuration configured for permitting sensing of a majority of said selected second portion.
76. The method of any one of embodiments 62-75, wherein said first sensing spots comprise said second sensing spots.
77. The method of any one of embodiments 62-76, comprising arranging said second sensing sports in a second array; each second sensing spot being positioned one with respect to the other along at least one second continuous line; wherein the method further comprising analyzing said second sensing data to identify a heat signature of one or more anatomical landmarks for verifying a good placement of said attachable sheet, and if no heat signature is identified, the method further comprising outputting a misplacement alert.
78. The method of embodiment 77, wherein said at least one second continuous line is lateral to said first selected portion.
79. The method of embodiment 77 or 78, wherein said at least one second continuous line is medial to said first selected portion.
80. The method of any one of embodiments 62-79, wherein said electrical profile is characterized by application of an electrical current, comprising said electrical current profile, in a continuous or discontinuous manner, or any combination thereof.
81. The method of any one of embodiments 62-80, wherein said at least one current electrode is configured for transmitting pulses of electrical current having a frequency in a range of about 1 kHz to about 10 MHz, with peak-to-peak voltage of about 0 to about 5 volt and with peak-to-peak current of about 40-70 micro Amper.
82. The method of any one of embodiments 62-81, comprising attaching a plurality of first pressure applicators, each configured for controllably applying pressure on an area of the breast or the axilla associated therewith;
   controlling said first pressure applicators to execute a selected first pressure plan on the breast and/or axilla; and
   sensing from a skin portion associated with the first sensing spots at least one of deformation profile, pressure profile or pressure change profile in response to controlled pressure application and generate respective data that is comprised in said first sensed data.
83. The method of embodiment 82, wherein the first sensing spots comprise a plurality of first pressure sensors.
84. The method of embodiment 82 or 83, wherein said selected first pressure plan comprises applying pressure simultaneously on selected mammary peripheral portions of the breast for a selected duration, followed by a coordinated relief of the pressure from said mammary peripheral portions; and
   operating said first sensing units to at least sense the temperature profile in response to said selected first pressure plan.
85. The method of embodiment 84, wherein said coordinated relief comprises relieving the pressure simultaneously from said mammary peripheral portions.
86. The method of embodiment 84 or 85, wherein said mammary peripheral portions form a continuum.
87. The method of any one of embodiments 84-86, wherein said application of pressure on said selected peripheral portions results in a continuous pressure application along a force application line.
88. The method of any one of embodiments 84-87, wherein said selected first pressure plan comprises:
   applying pressure with at least one first early pressure applicator and relieving the pressure application of said at least one first early pressure applicator,
   following the relief of pressure of the at least one first early pressure applicator, applying a pressure with at least one first subsequent pressure applicator and relieving the pressure application of said at least one first subsequent pressure applicator,
   wherein said at least one first subsequent pressure applicator is arranged spatially consecutively to said at least one first early pressure applicator; and
   the method further comprising operating said first sensing units to at least sense the deformation profile, pressure profile or pressure change profile in response to said selected first pressure plan.
89. The method of any one of embodiments 84-88, comprising analyzing said at least first sensed data and identifying therein signatures indicative of abnormality in at least one of said selected first portion and said selected second portion of the subject and generating abnormality data based thereon; and
   outputting said abnormality data.
90. The method of embodiment 89, further comprising receiving one or more user-related parameters indicative of at least one of: medical-related data of the subject; demographic data of the subject; genetic data of the subject; age; BMI; family history of the subject indicative of abnormalities of the breast, ovaries, and/or uterus; family history or personal history of the subject indicative of cancer and/or cancer-related pathologies; ethnicity; objective data of the subject, and; subjective data of the subject; and wherein said identifying is further based on said one or more user-related parameters.
91. The method of embodiment 89 or 90, wherein said analyzing further comprises generating a sensing map of at least one of deformation profile, pressure profile or pressure change profile in response to controlled pressure application based on said respective first data.
92. The method of any one of embodiments 89-91, wherein said analyzing comprises comparing each of said respective first sensed data to a baseline data for identifying a variation from said baseline being indicative of an abnormality.
93. The method of embodiment 92, wherein said baseline is selected from any one of: a respective sensed data of the contralateral breast and/or axilla, subjective tissue density of the examined breast, a pre-obtained sensed data of a subject having a certain degree of similarity to one or more said user-related parameters, other subjective and/or objective medical-related data adjusted to demographic data, such as age, BMI and ethnicity.
94. The method of any one of embodiments 89-93, wherein said analyzing comprises analysis of fat percentage of said first selected portion for determining said subjective tissue density.
95. The method of any one of embodiments 62-94, comprising applying a selected current profile between one or more pairs of current electrodes that are positioned in opposite sides of the breast, each member of said one or more pairs of electrodes is associated with a respective fat measuring electrode that is comprised in said plurality of measuring electrodes;
   wherein the method further comprises measuring electrical profile by the respective fat measuring electrodes, wherein said electrical profile measured by the fat measuring electrodes is indicative of the fat percentage in the tissue.
96. The method of any one of embodiments 62-95, wherein said plurality of measuring electrodes comprises said at least one current electrode.
97. The method of any one of embodiments 62-96, wherein each of said plurality of measuring electrodes functions also as a current electrode.
98. The method any one of embodiments 62-97, wherein said plurality of measuring electrodes are clustered to a plurality of groups, each group comprises a plurality of measuring electrodes members; wherein the method further comprises measuring an electrical profile between each measuring electrode member of a group to any of other measuring electrode members at the same group; wherein said electrical profile is selected from one or more of impedance, voltage and current.
99. The method of embodiment 98, wherein each measuring electrode is also a current electrode capable of applying an electric current to the tissue it is associated with; wherein for each measurement of electrical profile between each measuring electrode member of a group to any of other measuring electrode members, a selected profile of electric current is applied from one member participating in the measurement to another measurement participating in the measurement.
100. The method of embodiment 98, wherein the electrical profile between each measuring electrode member of a group to any of other measuring electrode members of the group is measured without application of electrical current to measure the natural electrical profile of the breast.
101. The method of any one of embodiments 62-100, wherein the first sensing spots comprise said second sensing spots.
102. The method of any one of embodiments 62-101, wherein said plurality of measuring electrodes are clustered to a plurality of circular groups, such that each measuring electrode in the circular group is at the same level as at least one other measuring electrode in the same group; wherein the method further comprises measuring electrical profile between one measuring electrode of the circular group and at least one other measuring electrode in the group.
103. The method of embodiment 102, wherein the method further comprises measuring electrical profile between one measuring electrode of the circular group and an opposite measuring electrode of the group positioned at an opposite side of a circle defining the arrangement of the circular group.
104. The examination unit of any one of embodiments 1-52, said at least one current electrode comprise one or more pairs of electrodes that are positioned in opposite sides of the attachable sheet, each member of said one or more pairs of electrodes is associated with a respective fat measuring electrode that is comprised in said plurality of measuring electrodes;
   wherein the processing circuitry is configured to operate the one or more pairs of electrodes to apply a selected current profile to flow from a first member electrode of the pair to a second member of the pair, the electrical profile that is sensed by the respective fat measuring electrodes is indicative of the fat percentage in the tissue.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic illustration of a non-limiting example showing some of the components of the system according to an aspect of the present disclosure.

FIGS. 2A-2B are schematic illustrations of a cross-sectional view of non-limiting examples of the structure of the breast unit and the armpit unit of the present disclosure.

FIG. 3 is a schematic illustration of a cross-sectional view of a non-limiting example of the configuration of the pressure applicators of the breast unit and the armpit unit of the present disclosure.

FIG. 4 is a schematic illustration of a non-limiting example of an arrangement of the pressure sensors.

FIG. 5 is a schematic illustration of a non-limiting example of an embodiment of the examination unit according to an aspect of the present disclosure.

FIG. 6 is a schematic illustration of a non-limiting example of a measurement scheme with the examination unit of FIG. 5.

FIG. 7 is a schematic illustration of a non-limiting example of the structure of examination unit that is composed of a flexible or stretchable PCT that is connected to a rigid PCB.

FIG. 8 is a schematic illustration of a non-limiting example of the system according to an aspect of the present disclosure.

FIGS. 9A-9B are schematic illustrations of non-limiting examples of electrical profile measurement by the examination unit according to an aspect of the present disclosure.

FIG. 10 is a schematic illustration of a non-limiting example of electrical profile measurement by the examination unit according to an aspect of the present disclosure.

FIG. 11 is a non-limiting exemplifying the shape of the attachable sheet of the examination unit according to an aspect of the present disclosure.

### DETAILED DESCRIPTION

The following figures are provided to exemplify embodiments and realization of the invention of the present disclosure.

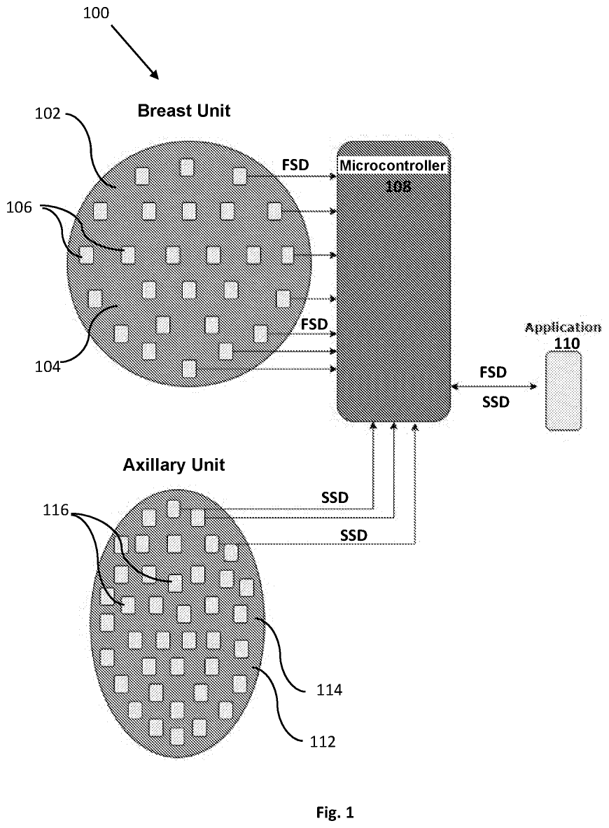

FIG. 1-3 are schematic illustrations of non-limiting examples of different components of a system for performing examination of a breast, an armpit or both of a subject and identifying abnormality in said breast, armpit, or both, according to an aspect of the present disclosure. The system 100 comprises a first, breast, unit 102 being configured for attachment to a portion of the breast of the subject. The first unit 102 comprises a first sheet 104 that comprises a plurality of first sensing units 106, each disposed at a different location on the sheet to allow sensing a different area of the breast when the first unit 102 is attached to the breast. Each first sensing unit 106 may include one or more types of sensors and allows sensing from a skin portion of the breast, that is associated therewith after the attachment of the first unit 102 to the breast, at least one of (i) deformation profile, pressure profile or pressure change profile, and (ii) IR radiation profile or temperature profile of the respective skin portion. Each first sensing unit 106 is configured to sense from a respective skin portion of the breast and generate respective first sensed data FSD based thereon. The first sensed data FSD includes a temporal profile of the measurements of the respective sensing unit 106 and is transmitted to a processing circuitry 108 or a microcontroller (the two terms are herein interchangeable). The processing circuitry 108 is configured to control the operation of the sensing units 106 and receive the first sensed data FSD generated by each of the first sensing units 106. The processing circuitry 108 is further configured for communicating with an analyzing module 110 that can be in the form of an application installed on a mobile unit or a computing module in the server. The processing circuitry 108 is configured to transmit each of the respective first sensed data FSD to the analyzing module 110, and the analyzing module 110 is configured to analyze each of the first sensed data FSD and to identify a signature that is indicative of an abnormality of the breast of the subject, such as a tumor or otherwise suspicious tissue structure/mass.

Each first sensing unit 106 may include a temperature sensor, a pressure sensor or a temperature sensor and a pressure sensor. Each first sensing unit 106 is associated with a different breast area of the subject and the sensed data that is collected in a specific sensing unit is related to the specific breast area it is associated with.

The system further comprises a second, armpit, unit 112 being configured for attachment to a portion of the armpit of the subject. While the system is described to include the second unit 112 it is to be noted that the system may exclude the second unit 112 such that the examination is performed only for the breast and not the armpit. The second unit 112 comprises a second sheet 114 that comprises a plurality of second sensing units 116, each disposed at a different location on the sheet to allow sensing a different area of the armpit when the second unit 112 is attached to the armpit. Each second sensing unit 116 includes at least a pressure sensor and allows sensing from a skin portion of the armpit, that is associated therewith after the attachment of the second unit 112 to the armpit, at least deformation profile, pressure profile or pressure change profile of the respective skin portion. Each second sensing unit 116 is configured to sense from a respective skin portion of the armpit and generate respective second sensed data SSD based thereon. The second sensed data SSD includes a temporal profile of the measurements of the respective sensing unit 116 and is transmitted to a processing circuitry 108 to be treated similarly to the first sensed data FSD. The processing circuitry 108 is further configured to control the operation of the second sensing units 116.

Each of the first and second units 102 and 112 further comprises first and second array of pressure applicators 118 and 120, respectively. The processing circuitry 108 is further configured for controlling the operation of the first and second array of pressure applicators 118 and 120 to execute a selected first and second pressure plans, respectively. Each sensing unit of the first and second sensing units 102 and 112 that is capable of sensing at least deformation profile, pressure profile or pressure change profile, is associated with a one or more pressure applicators that are configured to apply pressure on a skin portion of the subject, be it in the breast or the armpit, such that the effect of the pressure application is sensed by the respective sensing unit. In other words, pressure sensors of any of the first and second sensing units 102 and 112 are configured to sense the results of the application of pressure performed by pressure applicators on a skin portion they are associated with.

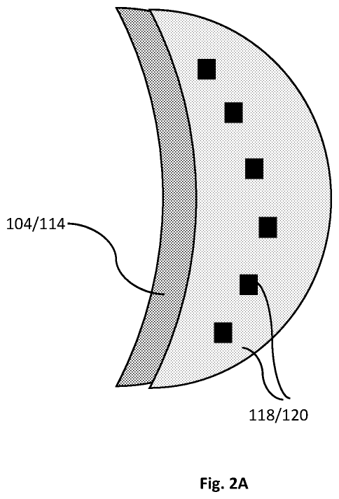

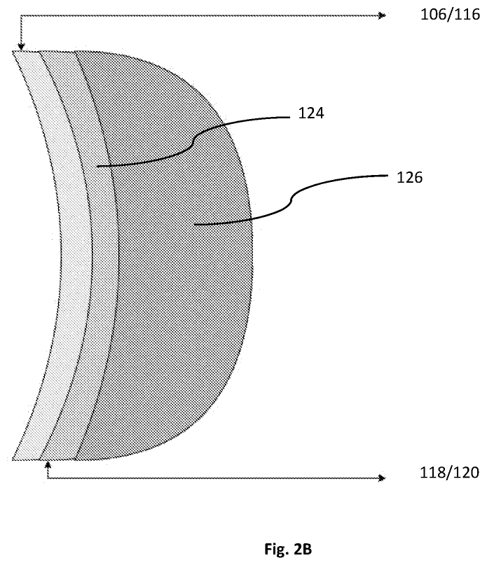

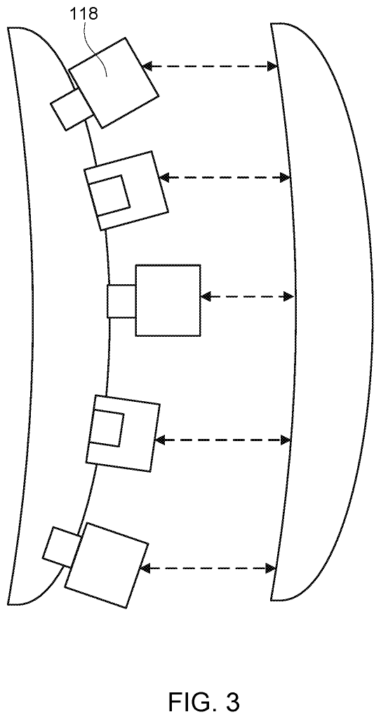

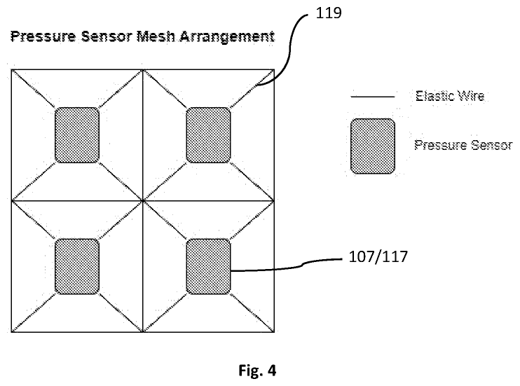

The first unit and the second unit may have a structure that comprises two or three layers. A two-layers structure is exemplified in FIG. 2A, and a three-layers structure is exemplified in FIG. 2B. The examples of FIGS. 2A-2B apply to either of the first and second unit. Reference is now being made to FIG. 2A, in which the unit 102/112 comprises a sheet 104/114 constituting the first layer and comprising the sensing units 106/116 (not shown specifically in FIGS. 2A-2B). The sensing units 106/116 are arranged such that one each selected area portion of the sheet there is at least one sensing unit. For example, on each 1 cm² or less there can be at least one sensing unit. The second layer 124 comprises the processing circuitry 108 and the pressure applicators 118/120 that are configured to apply pressure on a skin portion through the first layer. FIG. 3 shows an example of the pressure applicators 118/120 that are formed on the second layer and apply pressure on the first layer. The pressure applicators shown in FIG. 3 are at different states. Namely, FIG. 3 comes to exemplify that each pressure applicator can be independently controlled and that the pressure applicators can be in different states at the same time, each applying a different extent of pressure on the first layer that results in a different extent of pressure on the breast portions associated with the pressure applicators. Furthermore, FIG. 4 exemplifies a specific non-limiting example of an arrangement of the pressure sensors of the sensing units 106/116. Each pressure sensor is coupled with a plurality of own elastic wires 119 or elastic elements and each of the pressure sensors 107/117 is associated with a respective pressure applicator (not shown in FIG. 4) such that a pressure application of the respective pressure applicator results in an isolated deformation of elasticity of the elastic wires that are coupled to the respective pressure sensor, which in turn results in an isolated application of pressure on a specific breast or armpit portion. As can be appreciated, all elastic wires that are coupled to a respective pressure sensor are solely coupled to one pressure sensor to allow the isolation of pressure application on a specific breast or armpit portion and sensing its response to said isolated pressure application. This allows to isolate specific breast portion and measure their response to application of pressure thereon.

FIG. 2B shows an example in which the units 102/112 have a three-layers structure. The first layer is a sheet 104/114 comprising the sensing units 106/116 similar to FIG. 2A. The second layer 124 comprises the pressure applicators 118/120, and the third layer 126 comprises a the processing circuitry and wires connecting the processing circuitry to the different components in the first and second layers.

The system may include several unique examination schemes.

A first unique examination scheme is aimed at measuring the temporal temperature profile of areas of the breast after reducing an exchange of blood to or from tissues associated therewith. The first unique examination scheme initiates with reducing the exchange of blood to some areas of the breast by applying a peripheral application profile by selected peripheral pressure applicators of the first array of first pressure applicators 118. For example, the majority or every first pressure applicator 118 that is disposed at the peripheral portion of the first unit 102 is operated to apply a simultaneous peripheral pressure on areas of the breast associated with the periphery of the first unit 102. This simultaneous application of peripheral pressure results in a reduction of blood exchange from or to inner areas of the breast. After a selected duration of such simultaneous peripheral pressure application, the pressure is relieved, and the restriction of the blood exchange is removed. The first sensing units 106 that are located such that they sense the inner areas' temperature, sense the temporal temperature profile following the relief of the peripheral pressure. Abnormal tissues of the breast are expected to exhibit a temporal temperature profile that has at least one unique signature and by identifying the signatures of such temporal temperature profiles, these abnormal tissues can be identified.

A second unique examination scheme is aimed at generating a continuous map of a plurality of areas of the breast or the armpit of pressure-related parameters, i.e., parameters that derive from deformation profile, pressure profile or pressure change profile that is measured by the sensing unit associated with each breast area or portion. The second unique examination scheme comprises consecutive measurements of consecutive sensing units of the effect of application of pressure on a skin portion associated with the sensing units. In other words, the first and second pressure applicators are operated to apply a pressure according to a pressure application plan such that each session of the plan comprises application on a subsequent area of the breast or armpit that is shifted from the previous area that pressure was applied on. It is to be noted that the subsequent area may include a portion of the previous area. Therefore, the second scheme comprises operating at least one early pressure applicator and relieving the pressure application of said at least one first early pressure applicator. Following the relief of pressure of the at least one early pressure applicator, applying a pressure with at least one subsequent pressure applicator and relieving the pressure application of said at least one first subsequent pressure applicator. The at least one subsequent pressure applicator is arranged spatially consecutively to said at least one early pressure applicator. The sensing units are operated to sense the response of the skin portions on which pressure was applied by the early and the subsequent pressure applicators, thereby allowing the generation of a continuous map of pressure-related parameters. It is to be noted that the second examination scheme is applied to the first unit, the second unit or both, namely to the breast, the armpit or both.

Each of the first and second examination schemes are operated and controlled by the processing circuitry of the system.

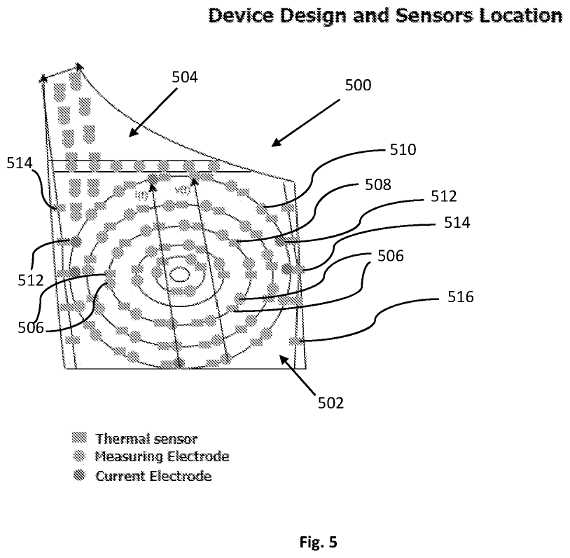

Reference is now being made to FIGS. 5-8, which are schematic illustrations of different components of non-limiting examples of embodiments of the examination unit and the system according to an aspect of the present disclosure. The examination unit 500 is configured as a patch that is intended to be attached to a breast portion of the subject, by a first part thereof 502, and to an axilla portion of the subject (i.e. the armpit), by a second part thereof 504. The first part 502 and the second part 504 are forming together a single examination unit 500 that is attached to the skin of the subject as one piece. The examination unit 500, whether it is a single patch or two patches, is configured to cover the whole area of the breast (Clavicle bone to mammary fold) and axilla. The examination unit 500 comprises a plurality of first sensing spots 506, each sensing spot is configured to sense from a tissue portion associated therewith either (i) electrical profile; (ii) IR radiation profile or thermal profile; or (iii) both electrical profile and IR radiation profile or thermal profile, and generate a respective first sensed data based thereon. The measurements of the electrical profile and the thermal profile are performed by thermal sensors 508 and measuring electrodes 510, respectively. The thermal sensors 508 can be of any type of known in the art thermal sensors, such as IR sensors, thermocouples or the like. The measuring electrodes are configured to measure one or more electrical parameters, such as current, voltage, impedance, permittivity, or any other electrical parameter. Based on the measurements of the thermal profile and the electrical profile, maps of the parameters are generated. Each map contains information of the parameter of every measured location of the skin of the subject along a selected time window. Namely, each map contains the measure of the parameter from a certain location at each time over the time of the measurement. Based on these maps, abnormality in the breast or the axilla can be identified in a post-processing analysis that can be carried out remotely from the examination unit, e.g. in the cloud or on a mobile device. Typically, each sensing spot 506 contains both thermal sensors 508 and measuring electrodes 510 such that the ratio of the thermal sensors and the measuring electrodes is 1:1 or about 1:1, thereby simplifying the correlation of the different maps. The first sensing spots 506 are arranged on one or more flexible PCBs and may be arranged in many ways. For example, some of the first sensing spots 506 can be arranged along concentric circles or can be arranged to form a spiral shape. Spiral shape configuration (not shown) is very advantageous as it allows a relatively close relations between adjacent sensing spots and allows relatively easy attachment to concave shapes such as the shape of the breast and adaptiveness of the device to the variety of different shapes of the chest area. Current electrodes 512 are arranged in different locations of the examination unit 500 and they are configured to execute, by an operation of a processing circuitry, an electric current profile to the skin of the subject such that a response to this electric current profile can be identified and measured by at least some, or in some embodiments all of the plurality of the first sensing spots 506. Typically, the current electrodes 512 are arranged in pairs on opposite sides of the examination unit 500. It is to be noted that the current electrodes 512 may be comprised in the first sensing spots 506, namely that some of the measuring electrodes may be also current electrodes. In some embodiments of the examination unit, one or two pairs of electrodes are placed such that each member of each pair is positioned separately from the other such that they are in two opposite locations of the examination unit. For example, two electrodes can be placed in the inferior mammary fold, and two in the superior border of the breast (i.e. 2 cm below the clavicle). The examination unit is configured to execute a current that flows between the electrodes (from the top of the breast to the bottom) providing a measurement of fat percentage in the tissue. The fat percentage of the breast tissue is calculated according to impedance data recorded in the electrodes placed in the superior and inferior borders of the breast that are in proximity to the current electrodes between which the electrical current flows. Using this parameter, the breast density and structure can be calculated and used in the analysis model that is performed in the present disclosure.

The examination unit 500 further comprises a plurality of second sensing spots 514, each comprises a thermal sensor 516 for measuring thermal profile of a skin portion associated with it. The second sensing spots 514 are arranged in one or more arrays that follows the anatomical structure of the main arteries in the breast—the lateral thoracic artery, and the internal mammary artery. The arrays of the second sensing spots 514 are arranged in a generally vertical orientation that follows the anatomical structure of these arteries, in particular following the scattering of the Lactiferous ducts and lobes. The second sensing spots 514 are configured to detect thermal signatures of these arteries to validate that the examination unit is adequately attached to the breast and axilla. If the thermal signatures of the arteries are not detected, then an alert is outputted to the user that there is a misplacement of the examination unit 500 and the user is requested to reattach the examination unit 500 in the correct position. This is repeated until the thermal signatures are detected by each array.

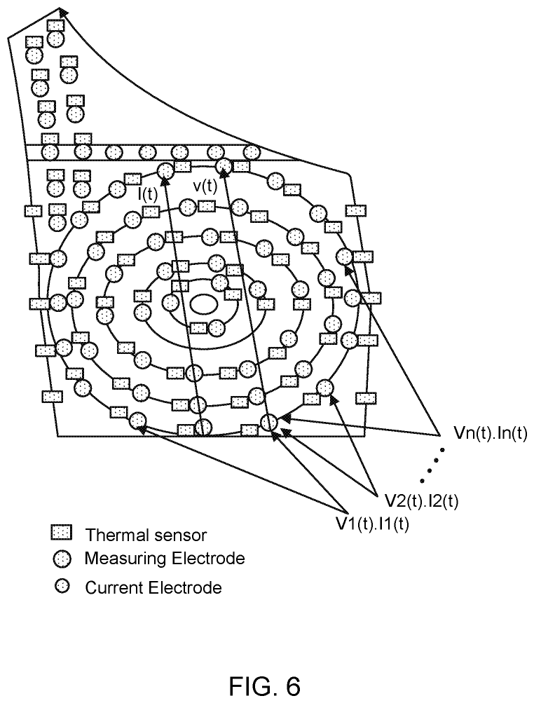

The first measurement that is carried out by the examination unit is the electrical profile measurement followed by the thermal profile measurement. The rationality of this is because the flow of the current in the breast tissue may cause an increase in blood vessels size, and thus affect the thermal sensing and may improve it. Typically, the electrical profile measurement is carried out between any two measuring electrodes of the first sensing spots 506, namely the current and the voltage are measured between any two pairs of measuring electrodes after the current electrodes 512 apply the selected electric current profile. This is exemplified by FIG. 6. Based on this electrical measurement, the impedance, the conductance and the permittivity are extracted. The frequency of the current wave in the electrodes, i.e. the electric current profile, may be from 1 khz to 10 Mhz, with voltage peak to peak of 0-5 v square, sinusoidal wave or other pulse and with peak-to-peak current of about 40-70 micro Amper. The frequency of the current may be personally determined and suited according to the subject's characteristics-Age, Breast size and volume, and tissue type. Both bra size and breast volume (which are calculated using a measurement of the length and width of the breast) may be used to determine impedance adjustments according to breast tissue density and fatty vs glandular components.

The breast tissue can be addressed to as a tissue with real impedance since the capacitive component is significantly smaller than the real impedance. The following equation can be used to calculate the Real impedance part within the breast tissue:

$$\frac{ρ}{4 π} [ \frac{1}{{R}_{1}} - \frac{1}{{R}_{2}} ] .$$

R₁—Nipple diameter divided by 2; R₂—Breast base diameter divided by 2; ρ-Breast tissue density which is calculated using the breast volume measurement.

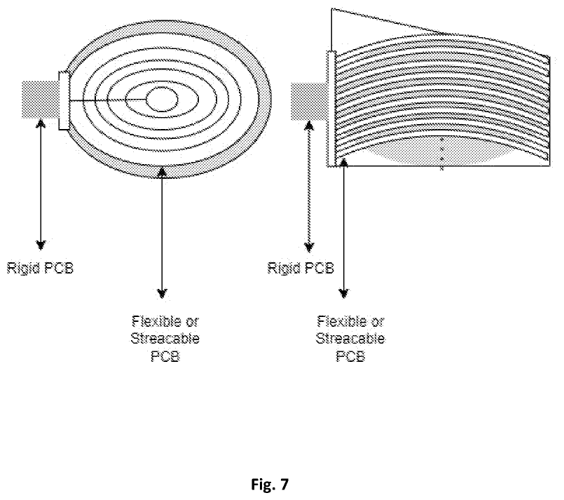

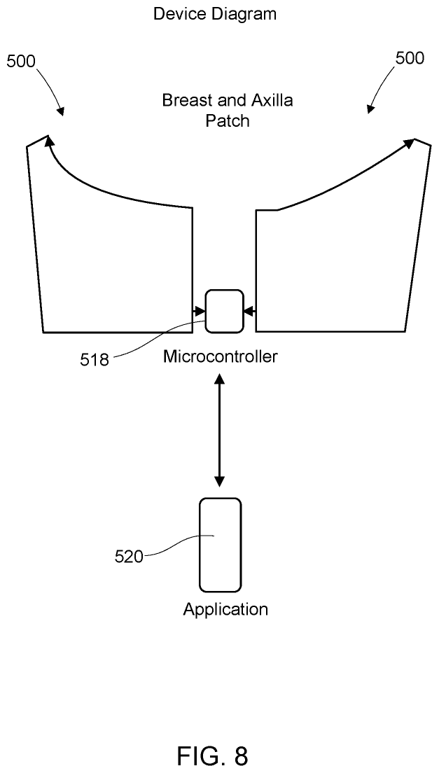

A processing circuitry 518 is comprised in the examination unit 500, as exemplified in FIG. 8, and is configured to control the operation of the current electrode and the sensors of the sensing spots, and to transmit the collected sensed data to a remote device or processor 520, such as to the cloud or to a mobile device. It is to be noted that each side of the breast has its own examination unit and each may have its own processing circuitry that operates the entire measurement. Alternatively, one processing circuitry in one of examination units may operate the two examination units (the other receives the commands wirelessly). FIG. 7 exemplifies the integration of the processing circuitry 518 to the flexible or stretchable PCB that comprise the first and second sensing spots and the current electrodes. A rigid PCB connects the processing circuitry, the sensing spots, the current electrodes and a communication module (not shown) together. The communication module may be part of the processing circuitry or independent therefrom and is configured to transmit the sensed data, either raw or processed, to the remote device 520, e.g. a cloud or a mobile device, to thereby undergo further analysis to generate the maps, correlate them and identify based thereon whether there is an abnormality. For example, malignant tissues may exhibit a variation in their capacitive component with respect to healthy tissues. Analysis on the generated maps can identify such variation.

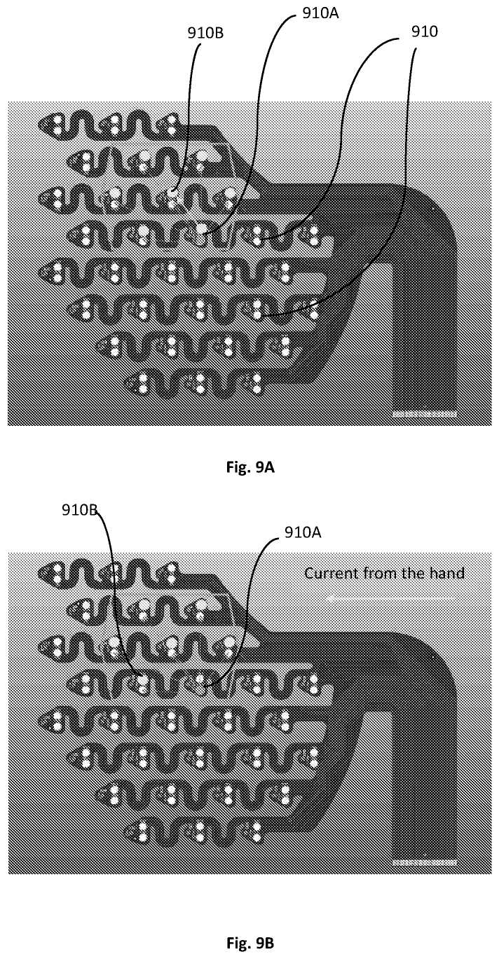

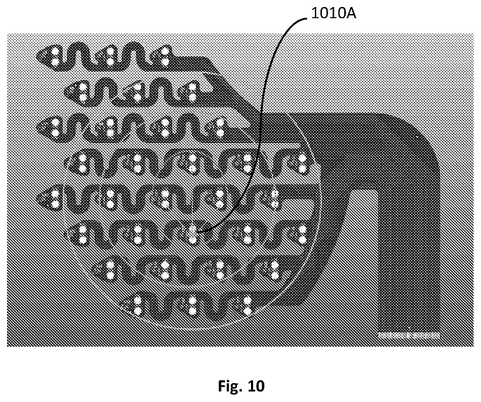

Reference is now being made to FIGS. 9A-9B, which are schematic illustrations of non-limiting examples of electrical profile measurement by the examination unit according to an aspect of the present disclosure. The figures present similar concept of a PCB that has a plurality of branches, wherein each branch comprises windings and the measuring electrodes 910 are positioned along these windings. The measuring electrodes 910 are clustered into groups of electrodes. In these examples, the group includes 7 measuring electrodes that are arranged in a hexagon, which is shown in the figures for ease of explanation. It is to be noted that the groups can include different numbers of sensors and can be arranged in different shapes. The groups are selected to have some degree of overlap between them and the measuring electrodes that are associated with areas of the breast that are more prone to include cancer are included in the highest number of groups. During the measurement, the electrical profile is measured between each measuring electrode of the group and any other measuring electrode member of the same group. For example in the figures, the electrical profile between electrode 910A and any other electrode within the hexagon is measured. The electrical profile is either voltage, current, impedance or any combination thereof. The difference between FIG. 9A and FIG. 9B is the source of the electrical current that is applied to the tissue during the measurement. In FIG. 9A the electrical current is applied from the measuring electrode from which the electrical profile is measured with respect to any other electrode in the group. In the example of FIG. 9A, the measuring electrode 910A serves as a current electrode and applies electrical current profile towards the electrode that participates in the measurement. For example, when the electrical profile between electrode 910A and electrode 910B is measured, electrical current profile is applied from electrode 510A towards electrode 910B. In FIG. 9B, the electrical current profile is always applied on the hand of the subject and spreads uniformly over the breast according to the resistance of the tissue.

Reference is made to FIG. 10, which exemplifies a similar arrangement of measuring electrodes as in FIGS. 9A-9B, but a different measuring method. FIG. 10 exemplifies clustering of measuring electrodes along lines, in this example circles, each defining a certain level of the all the measuring electrodes on it with respect to the breast profile. In other words, each line defines a certain height difference from the peak of the breast, i.e. the nipple. Therefore, the shortest way between each two measuring electrodes passes through a deep portion of the breast tissue, and this is in particular relevant when the measurement is made between two opposite measuring electrodes of a circle. This allows to identify abnormalities that may be found in deep breast tissues. For example, the measurement is performed between electrode 1010A and any other electrode on the most inner circle. This measurement can be done by any one of the techniques described in FIG. 9A-9B, namely when the electrical current is either applied from the measuring electrode or from the hand.

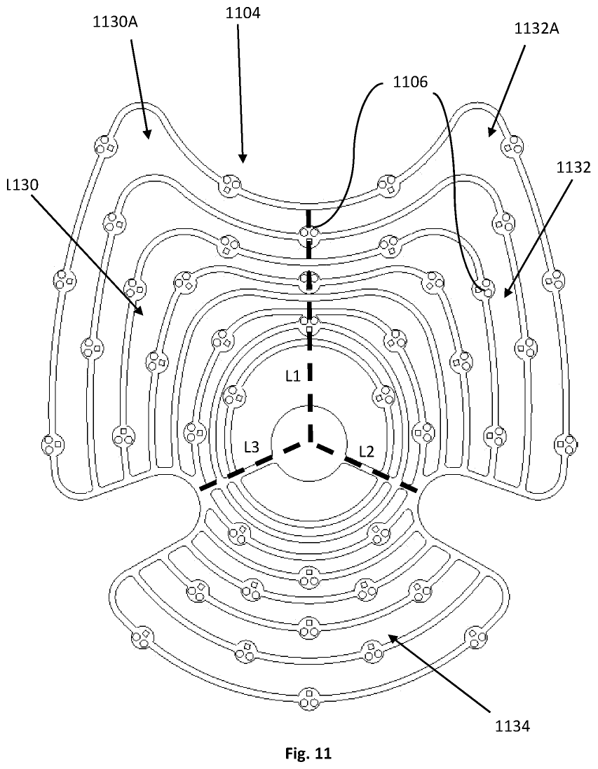

Reference is now being made to FIG. 11, which is a non-limiting exemplifying the shape of the attachable sheet of the examination unit according to an aspect of the present disclosure. The attachable sheet 1104 comprises a plurality of sensing spots 1106, each sensing spot comprises either (1) an IR or temperature sensor, a measuring electrode that is configured to measure electrical profile of a skin portion it is associated with, or (3) both IR or temperature sensor and measuring electrode. The attachable sheet 1104 is formed of three flap-like or wing-like portions, a first portion 1130, a second portion 1132 and a third portion 1134. The first and second portions 1130 and 1132 are shaped to conform with the shoulder or upper arm portion of the subject such that the first portion 1130 partially encircles partially the shoulder or the upper arm portion from one side and the second portion 1132 partially encircles from the other side such that part of their respective distal portions 1130A, 1132A overlap or in proximity when are attached properly to the body. The third portion 1134 has a certain degree of freedom with respect to the first and second portions 1130, 1132 to allow adjustment of the attachable sheet 1104 to conform with the different shapes and sizes of breasts. For example, the third portion can be brough into proximity or even to overlap with either the first or the second portions 1130, 1132 to conform with the shape of a relatively small breast. It is to be noted that there is no clear discernable division between the three portions, however they can be discerned by the periphery profile of the attachable sheet 1104. The periphery profile is defined by a varying distance from the nipple-intended portion 1136 of the attachable sheet 1104, that is located at about the center of the attachable sheet 1104. The transition between one portion to another can be defined by the line connecting the nipple-intended portion 1136 and the part on the periphery which is closest to the nipple-intended portion 1136, namely the part which has at least the local minimum distance to the nipple-intended portion 1136. These lines are exemplified in the figure by dashed lines L1, L2 and L3.

It is to be noted that the embodiments of FIGS. 1-4 can be combined with the embodiments of FIGS. 5-11, to yield an examination unit that is capable of measuring thermal profile, electrical profile and pressure response profile.

1-44. (canceled)

45. An examination unit for performing an examination of at least one of a breast of a subject and an axilla of the subject and for identifying a tissue abnormality, the examination unit comprising:
at least one sheet to be attached to the at least one of the breast of the subject and the axilla of the subject, the at least one sheet comprising
a plurality of measuring electrodes,
a plurality of first sensing spots, each first sensing spot associated with a tissue portion and configured to
sense at least one of an electrical profile by a respective measuring electrode from the plurality of measuring electrodes, and an IR radiation profile or thermal profile, and
generate a respective first sensed data based sensed data,
a plurality of second sensing spots, each second sensing spot configured to sense from a tissue portion associated therewith an IR radiation profile or thermal profile and generate a respective second sensed data based thereon, and
at least one current electrode configured to apply electrical current to at least a tissue portion associated with a respective the first sensing spot;
a processor and memory cooperating therewith and configured to
operate the first and second sensing spots and receiving each of the respective first and second sensed data,
control the at least one current electrode to execute a selected electrical current profile, and
transmit each of the respective first and second sensed data;
wherein the plurality of measuring electrodes is clustered to a plurality of groups, each group comprises a plurality of measuring electrodes members;
wherein the processor is configured to operate the plurality of measuring electrodes to measure an electrical profile between each measuring electrode member of a group to any of other measuring electrode members at the same group;
wherein the electrical profile is selected from one or more of impedance, voltage and current; and
wherein some of the measuring electrodes are common to two or more groups.

46. The examination unit of claim 45 wherein the at least one sheet comprises a skin facing face, the skin facing face comprising an adhesive layer for allowing attachment to the first and second selected portions; and wherein the skin facing face comprises a removable cover covering the adhesive layer and to be removed prior to the attachment.

47. The examination unit of claim 45 wherein at least a portion of the measuring electrodes constitute the at least one current electrode.

48. The examination unit of claim 45 wherein the electrical profile is determined by at least one of impedance, conductivity, voltage, current, and permittivity of the at least the tissue portion being associated with a respective first sensing spot thereto.

49. The examination unit of claim 45 wherein each of the plurality of first sensing spots comprises a measuring electrode and a thermal sensor; and wherein the first sensing spot is configured to sense at least one of an electrical profile, an IR radiation profile, and a thermal profile of the respective tissue portion associated therewith.

50. The examination unit of claim 45 comprising at least one optical sensor on different portions of a periphery of the at least one sheet and configured to sense optical parameters indicative of light intensity of one or more selected wavelength ranges and generating optical sensed data based thereon; wherein the processor is configured to analyze the optical sensed data to identify insufficient level of adhesiveness or adherence of the at least one sheet to the skin of the subject and outputting an alert if the insufficient level of adhesiveness is identified.

51. The examination unit of claim 45 wherein the plurality of first sensing spots comprises the plurality of second sensing spots.

52. The examination unit of claim 51 further comprising a second sensing array having a plurality of the second sensing spots; wherein each second sensing spot is positioned one with respect to the other along at least one second continuous line; wherein the processor is configured to analyze the second sensing data and to identify a heat signature of one or more anatomical landmarks for verifying a good placement of the attachable sheet, and if no heat signature is identified, the processor is configured to execute a misplacement alert; and wherein the at least one second continuous line is lateral or medial to the first selected portion.

53. The examination unit of claim 45 further comprising a plurality of first pressure applicators associated with the at least one sheet, each configured to apply pressure on an area of the at least one of the breast of the subject and the axilla of the subject associated therewith when the at least one sheet is attached to the breast;
wherein the first sensing spots are configured to sense from a skin portion associated therewith at least one of deformation profile, pressure profile or pressure change profile in response to controlled pressure application and generate respective data that is comprised in the first sensed data; and
wherein the processor is configured to control the first pressure applicators to execute a selected first pressure plan on the at least one of the breast of the subject and the axilla of the subject;
wherein the first sensing spots comprise a plurality of first pressure sensors;
wherein each first sensing spot that comprises a first pressure sensor is associated with a respective first pressure applicator, such that the respective first pressure applicator is configured to apply pressure on at least the breast area associated with the respective first sensing spot.

54. The examination unit of claim 53 wherein the selected first pressure plan comprises applying pressure simultaneously on selected mammary peripheral portions of the breast for a selected duration, followed by a coordinated relief of the pressure from the mammary peripheral portions;
wherein the processor is configured to operate the first sensing units to at least sense the temperature profile in response to the selected first pressure plan;
wherein the coordinated relief comprises relieving the pressure simultaneously from the mammary peripheral portions;
wherein the mammary peripheral portions form a continuum;
wherein the application of pressure on the selected peripheral portions results in a continuous pressure application along a force application line; and
wherein the selected first pressure plan comprises:
applying pressure with at least one first early pressure applicator and relieving the pressure application of the at least one first early pressure applicator,
following the relief of pressure of the at least one first early pressure applicator,
applying a pressure with at least one first subsequent pressure applicator, and
relieving the pressure application of the at least one first subsequent pressure applicator;
wherein the at least one first subsequent pressure applicator is arranged spatially consecutively to the at least one first early pressure applicator; and
wherein the processor is configured to operate the first sensing spots to at least sense the deformation profile, pressure profile or pressure change profile in response to the selected first pressure plan.

55. The examination unit of claim 45 wherein each measuring electrode is also a current electrode capable of applying an electric current to the tissue it is associated with; wherein the processor is configured to control each measuring electrode such that for each measurement of electrical profile between each measuring electrode member of a group to any of other measuring electrode members, a selected profile of electric current is applied from one member participating in the measurement to another measurement participating in the measurement.

56. The examination unit of claim 45 wherein the at least one sheet comprises a first portion, a second portion and a third portion; wherein the first portion and the second portion are shaped to fit over the shoulder of the subject, each from a different side of shoulder, so as to ensure a right placement of the at least one sheet over the at least one of the breast of the subject and the axilla of the subject; and wherein the third portion provides a degree of freedom with respect to the first and second portions to allow adjustment of the at least one sheet to different sizes and shapes of breasts.

57. The examination unit of claim 56 wherein each of the portions extends peripherally from a central portion of the at least one sheet; wherein the at least one sheet defines a varying periphery length from a central point of the at least one sheet to any point along the periphery of the at least one sheet; and wherein the peripheral length between two adjacent portions decrease to a certain minimum defining the border of the two adjacent portions.

58. The examination unit of claim 45 wherein the plurality of measuring electrodes is clustered to a plurality of circular groups, such that each measuring electrode in the circular group is at the same level as at least one other measuring electrode in the same group; wherein the processor is configured to operate the measuring electrodes to measure electrical profile between one measuring electrode of the circular group and at least one other measuring electrode in the group; and wherein the processor is configured to operate the measuring electrodes to measure electrical profile between one measuring electrode of the circular group and an opposite measuring electrode of the group positioned at an opposite side of a circle defining the arrangement of the circular group.

59. A system for performing examination of at least one of a breast of a subject and an axilla of the subject for identifying a tissue abnormality, the system comprising:
an examination unit comprising
at least one sheet to be attached to the at least one of the breast of the subject and the axilla of the subject, the at least one sheet comprising
a plurality of measuring electrodes,
a plurality of first sensing spots, each first sensing spot associated with a tissue portion and configured to
sense at least one of an electrical profile by a respective measuring electrode from the plurality of measuring electrodes, and an IR radiation profile or thermal profile, and
generate a respective first sensed data based sensed data,
a plurality of second sensing spots, each second sensing spot configured to sense from a tissue portion associated therewith an IR radiation profile or thermal profile and generate a respective second sensed data based thereon, and
at least one current electrode configured to apply electrical current to at least a tissue portion associated with a respective the first sensing spot,
a processor and memory cooperating therewith and configured to
operate the first and second sensing spots and receiving each of the respective first and second sensed data,
control the at least one current electrode to execute a selected electrical current profile, and
transmit each of the respective first and second sensed data,
wherein the plurality of measuring electrodes is clustered to a plurality of groups, each group comprises a plurality of measuring electrodes members,
wherein the processor is configured to operate the plurality of measuring electrodes to measure an electrical profile between each measuring electrode member of a group to any of other measuring electrode members at the same group,
wherein the electrical profile is selected from one or more of impedance, voltage and current, and
wherein some of the measuring electrodes are common to two or more groups;
an analyzing module configured to
receive and analyze the at least first sensed data and identify signatures indicative of abnormality in the at least one of the breast of the subject and the axilla of the subject, and
generate abnormality data based thereon; and
an output module configured to output the abnormality data.

60. The system of claim 59 wherein the analyzing comprises to analyze the second sensing data to identify a heat signature of one or more anatomical landmarks for verifying a good placement of the attachable sheet, and if no heat signature is identified, the processor is configured to execute a misplacement alert; wherein the analyzing further comprises generating a sensing map of at least one of deformation profile, pressure profile or pressure change profile in response to controlled pressure application based on the respective first data.

61. The system of claim 59 wherein the analyzing comprises comparing each of the respective first sensed data to a baseline data for identifying a variation from the baseline being indicative of an abnormality; wherein the baseline is selected from any one of: a respective sensed data of a contralateral breast and/or axilla, subjective tissue density of the examined breast, a pre-obtained sensed data of a subject having a certain degree of similarity to one or more the user-related parameters, other subjective and/or objective medical-related data adjusted to demographic data, such as age and ethnicity; wherein the analyzing comprises analysis of fat percentage of the first selected portion for determining the subjective tissue density; and wherein the analyzing module is configured to compare between first sensed data of two identical groups of first sensing spots, each measured in a different breast of the subject.

62. A method for performing examination of at least one of a breast of a subject and an axilla of the subject, the method comprising:
attaching a plurality of first sensing spots, a plurality of measuring electrodes, and at least one current electrode to the at least one of the breast of the subject and the axilla of the subject;
wherein each first sensing spot is associated with a tissue portion and is configured to
sense at least one of an electrical profile by a respective measuring electrode from the plurality of measuring electrodes, and an IR radiation profile or thermal profile, and
generate a respective first sensed data based sensed data;
wherein the at least one current electrode is configured to apply electrical current to at least a tissue portion associated with a respective the first sensing spot;
controlling the at least one current electrode to execute a selected electrical current profile;
operating the plurality of first and second sensing spots and receiving each of the respective first and second sensed data;
transmitting each of the respective first sensed data;
wherein the plurality of measuring electrodes is clustered to a plurality of groups, each group comprises a plurality of measuring electrodes members; and
measuring an electrical profile between each measuring electrode member of a group to any of other measuring electrode members at the same group;
wherein the electrical profile is selected from one or more of impedance, voltage and current; and
wherein some of the measuring electrodes are common to two or more groups.

63. The method of claim 62 further comprising
attaching a plurality of second sensing spots to the at least one of the breast of the subject and the axilla of the subject; wherein each second sensing spot configured to sense from a tissue portion associated therewith an IR radiation profile or thermal profile and generate a respective second sensed data based thereon; and
optically sensing optical parameters indicative of light intensity of at least one wavelength range between either the plurality of first sensing spots, the plurality of second sensing spots, or the plurality of first sensing spots and the plurality of second sensing spots, and the skin of the subject and generating optical sensed data; and
identifying in the sensed data an inadequate adhesiveness or adherence of the plurality of first sensing spots or the plurality of second sensing spots to the skin and outputting an alert if inadequate adhesiveness or adherence is identified.

64. The method of claim 62 further comprising:
applying a selected current profile between one or more pairs of current electrodes that are positioned in opposite sides of the breast, each member of the one or more pairs of electrodes is associated with a respective fat measuring electrode that is comprised in the plurality of measuring electrodes; and
measuring electrical profile by the respective fat measuring electrodes;
wherein the electrical profile measured by the fat measuring electrodes is indicative of the fat percentage in the tissue.
